SEQUENCE LISTING (10 sequences)

SEQ ID NO: 1 - DNA, 1440 bp, Artificial Sequence
  Human TSG-6
cagtcacatttcagccactgctctgagaatttgtgagcagcccctaacaggctgttacttcactacaactgacgatatgatcatcttaatttacttatttctcttgctatgggaagacactcaaggatggggattcaaggatggaatttttcataactccatatggcttgaacgagcagccggtgtgtaccacagagaagcacggtctggcaaatacaagctcacctacgcagaagctaaggcggtgtgtgaatttgaaggcggccatctcgcaacttacaagcagctagaggcagccagaaaaattggatttcatgtctgtgctgctggatggatggctaagggcagagttggataccccattgtgaagccagggcccaactgtggatttggaaaaactggcattattgattatggaatccgtctcaataggagtgaaagatgggatgcctattgctacaacccacacgcaaaggagtgtggtggcgtctttacagatccaaagcaaatttttaaatctccaggcttcccaaatgagtacgaagataaccaaatctgctactggcacattagactcaagtatggtcagcgtattcacctgagttttttagattttgaccttgaagatgacccaggttgcttggctgattatgttgaaatatatgacagttacgatgatgtccatggctttgtgggaagatactgtggagatgagcttccagatgacatcatcagtacaggaaatgtcatgaccttgaagtttctaagtgatgcttcagtgacagctggaggtttccaaatcaaatatgttgcaatggatcctgtatccaaatccagtcaaggaaaaaatacaagtactacttctactggaaataaaaactttttagctggaagatttagccacttataaaaaaaaaaaaaaggatgatcaaaacacacagtgtttatgttggaatcttttggaactcctttgatctcactgttattattaacatttatttattatttttctaaatgtgaaagcaatacataatttagggaaaattggaaaatataggaaactttaaacgagaaaatgaaacctctcataatcccactgcatagaaataacaagcgttaacattttcatatttttttctttcagtcatttttctatttgtggtatatgtatatatgtacctatatgtatttgcatttgaaattttggaatcctgctctatgtacagttttgtattatactttttaaatcttgaactttataaacattttctgaaatcattgattattctacaaaaacatgattttaaacagctgtaaaatattctatgatatgaatgttttatgcattatttaagcctgtctctattgttggaatttcaggtcattttcataaatattgttgcaataaatatccttgaacacaaaaaaaaaaaaaaaaaa

SEQ ID NO: 2 - protein, 277 aa, Artificial Sequence
  Human TSG-6
MIILIYLFLLLWEDTQGWGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 3 - protein, 260 aa, Artificial Sequence
  Human TSG-6
WGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 4 - DNA, 1440 bp, Artificial Sequence
  Human TSG-6
cagtcacatttcagccactgctctgagaatttgtgagcagcccctaacaggctgttacttcactacaactgacgatatgatcatcttaatttacttatttctcttgctatgggaagacactcaaggatggggattcaaggatggaatttttcataactccatatggcttgaacgagcagccggtgtgtaccacagagaagcacggtctggcaaatacaagctcacctacgcagaagctaaggcggtgtgtgaatttgaaggcggccatctcgcaacttacaagcagctagaggcagccagaaaaattggatttcatgtctgtgctgctggatggatggctaagggcagagttggataccccattgtgaagccagggcccaactgtggatttggaaaaactggcattattgattatggaatccgtctcaataggagtgaaagatgggatgcctattgctacaacccacacgcaaaggagtgtggtggcgtctttacagatccaaagcggatttttaaatctccaggcttcccaaatgagtacgaagataaccaaatctgctactggcacattagactcaagtatggtcagcgtattcacctgagttttttagattttgaccttgaagatgacccaggttgcttggctgattatgttgaaatatatgacagttacgatgatgtccatggctttgtgggaagatactgtggagatgagcttccagatgacatcatcagtacaggaaatgtcatgaccttgaagtttctaagtgatgcttcagtgacagctggaggtttccaaatcaaatatgttgcaatggatcctgtatccaaatccagtcaaggaaaaaatacaagtactacttctactggaaataaaaactttttagctggaagatttagccacttataaaaaaaaaaaaaaggatgatcaaaacacacagtgtttatgttggaatcttttggaactcctttgatctcactgttattattaacatttatttattatttttctaaatgtgaaagcaatacataatttagggaaaattggaaaatataggaaactttaaacgagaaaatgaaacctctcataatcccactgcatagaaataacaagcgttaacattttcatatttttttctttcagtcatttttctatttgtggtatatgtatatatgtacctatatgtatttgcatttgaaattttggaatcctgctctatgtacagttttgtattatactttttaaatcttgaactttataaacattttctgaaatcattgattattctacaaaaacatgattttaaacagctgtaaaatattctatgatatgaatgttttatgcattatttaagcctgtctctattgttggaatttcaggtcattttcataaatattgttgcaataaatatccttgaacacaaaaaaaaaaaaaaaaaa

SEQ ID NO: 5 - protein, 277 aa, Artificial Sequence [sequence omitted]

SEQ ID NO: 6 - protein, 260 aa, Artificial Sequence
  Human TSG-6
WGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKRIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 7 - protein, 92 aa, Artificial Sequence
  Human TSG-6
VYHREARSGKYKLTYAEAKAVCEFEGGHLATYKQLEAARKIGFHVCAAGWMAKGRVGYPIVKPGPNCGFGKTGIIDYGIRLNRSERWDAYCY

SEQ ID NO: 8 - DNA, 319 bp, Artificial Sequence
  Human TSG-6
aggagatatacatatgggtgtgtaccaccgtgaagcacggtctggcaaatacaagctcacctacgcagaagctaaggcggtgtgtgaatttgaaggcggccatctcgcaacttacaagcagctagaggcagcccgtaaaattggatttcatgtctgtgctgctggatggatggctaagggccgtgttggataccccattgtgaagccagggcccaactgtggatttggaaaaactggcattattgattatggaatccgtctcaataggagtgaacgttgggatgcctattgctacaacccacacgcaaagtaagaattc

SEQ ID NO: 9 - protein, 98 aa, Artificial Sequence
  Human TSG-6
GVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAK

SEQ ID NO: 10 - protein, 99 aa, Artificial Sequence
  Human TSG-6
MGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAK

(19) United States
(12) Patent Application Publication
(10) Pub. No.: US 2025/0121028 A1
(43) Pub. Date: Apr. 17, 2025

(54) USE OF LINK-TSG6 FOR THE TREATMENT OF OSTEOARTHRITIC PAIN

(71) Applicant: The University of Manchester, Manchester, Greater Manchester (GB)

(72) Inventors: Anthony Day, Manchester, Greater Manchester (GB); Caroline Milner, Manchester, Greater Manchester (GB)

(21) Appl. No.: 18/262,113

(22) PCT Filed: Jan. 19, 2022
(86) PCT No.: PCT/EP2022/051101

§ 371 Date: Jul. 19, 2023

(30) Foreign Application Priority Data
Jan. 20, 2021 (GB) 2100761.2

(51) Int. Cl. A61K 38/17; A61K 9/00; A61P 29/00

(52) U.S. Cl. CPC A61K 38/1709; A61K 9/0019; A61P 29/00

(57) ABSTRACT

This invention relates to the treatment of osteoarthritic pain, or pain associated with osteoarthritis, and particularly for LINK_TSG6 polypeptide for use in the treatment of osteoarthritic pain. Osteoarthritis is caused by the deterioration of cartilage in joints and is characterised by pain and a loss of mobility in the affected joints. Osteoarthritic pain results from multiple factors including bone marrow lesions, sub-articular bone attrition, synovitis, effusion and innervation of usually insensitive tissues. LINK_TSG6 is a short recombinant peptide comprising the LINK module of human TSG-6.

### RELATED APPLICATIONS

This application is the U.S. National Stage of International Application No. PCT/EP2022/051101, filed 19 Jan. 2022, which designates the U.S., published in English, and claims priority under 35 U.S.C. § 119 or 365(c) to Great Britain Application No. GB 2100761.2, filed 20 Jan. 2021. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file:
a) File name: Sequence_listing.txt; created Jul. 24, 2023, 17,296 Bytes in size.

### FIELD OF THE INVENTION

The present invention relates to the treatment of osteoarthritic pain and particularly, although not exclusively, to the treatment of osteoarthritic pain with a LINK_TSG6 polypeptide.

### BACKGROUND

Osteoarthritis (OA) is the most common form of arthritis worldwide. It is a long-term chronic disease caused by the deterioration of cartilage in joints and is characterised by pain and a loss of mobility in the affected joints (e.g. in the knee, hip and hand). The World Health Organization (WHO) estimates that 9.6% of men and 18% of women aged over 60 years have symptomatic OA, 80% of those will have limitations in movement, and 25% cannot perform their major daily activities of life. Multiple factors can contribute towards the development of OA, including age, sex, obesity, lack of exercise, genetic factors, bone density, prior joint injury or trauma, or joint disease such as gout or rheumatoid arthritis. OA prevalence is increasing due to population aging and also an increase in related factors such as obesity.

There is no disease modifying therapy for OA and current treatments are limited to pain relief and surgical intervention, primarily joint replacement. OA patients are having joint replacements at ever-younger ages, necessitating greater numbers of revision surgeries that typically have poor outcomes and require pain management.

Existing pain treatments for OA include acetaminophen (paracetamol), opiate analgesics (painkillers) including codeine and Nonsteroidal Anti-Inflammatory Drugs (NSAIDs). All of these have limitations or side effects. Acetaminophen can cause liver damage and is not suitable for those with existing liver conditions. Although narcotic analgesics are effective at treating moderate to severe OA pain, they can result in side effects including dizziness and nausea and may be habit-forming. NSAIDs can reduce swelling and inflammation caused by OA in addition to providing pain relief, however taking NSAIDs increases risk of GI problems and heart attack, stroke and heart failure, therefore, as reviewed in Altman and Barthel (Drugs. 2011; 71(10):1259-1279) it is often recommended that oral NSAIDs are prescribed at the lowest effective dose, for the shortest amount of time.

Topical treatments for OA pain include capsaicin cream that treats pain by causing reversible nerve degeneration. However, capsaicin can cause significant pain early in treatment and this may lead to discontinuation; a review found that of 1,556 subjects, 54% of patients treated with topical capsaicin experienced adverse reactions (compared to 15% of patients treated with placebo), and 13%, of patients treated with capsaicin discontinued treatment due to these effects (Altman and Barthel, Drugs. 2011; 71(10):1259-1279). In addition, there is a risk of side effects due to nerve damage, particularly for diabetic patients.

Steroid injections can be used to treat OA pain by reducing swelling and inflammation. These can be administered intra-articularly (into the joint), however some evidence is emerging that intra-articular steroid injections can cause side effects such as accelerated OA progression and bone loss (Kompel et al., Radiology. 2019; 293(3):656-663).

New treatments for alleviating osteoarthritic pain are therefore needed.

Yang et al., 2020 (J Neuroinflammation 17(1):154) describe the use of intrathecally injected Bone Marrow Stem Cells (BMSCs) that secrete TSG-6 and relieve neuropathic pain caused by chronic constriction injury in rats. This study also showed that intrathecal injection of exogenous TSG-6 attenuates CCI-induced neuropathic pain.

The present invention has been devised in light of the above considerations.

### SUMMARY OF THE INVENTION

The present disclosure relates to LINK_TSG6 polypeptide for use in the treatment of osteoarthritic pain. As used herein, the abbreviation OA refers to osteoarthritis, and OA pain refers to osteoarthritic pain, or pain associated with osteoarthritis.

As shown in the examples, LINK_TSG6 is capable of reducing osteoarthritic pain in a dose dependent manner. LINK_TSG6 was effective at reducing osteoarthritic pain when administered subcutaneously and intra-articularly at both high and low dosages, and at both monthly and weekly dosing regimens.

Administration of LINK_TSG6 reduced OA pain in a partial medial meniscectomy model of OA and OA pain, as shown by reduced ‘direct pain’ caused by flexion of the injured joint, improved thermal sensitivity and mechanical sensitivity, and improved weight bearing.

The analgesic effects of LINK_TSG6 were observed in the ipsilateral limb (i.e. the surgically operated side), but also in the contralateral side (i.e. the opposite side of the body to the surgically operated limb). This indicates that LINK_TSG6 has analgesic properties, and may be useful for administration both directly to the affected osteoarthritic joint and at more distant sites. Moreover, LINK_TSG6 may be suitable for the treatment of referred pain, such as pain in locations distant to an osteoarthritic joint, as well as being suitable for direct pain resulting from osteoarthritis in a joint.

In a first aspect, the disclosure provides LINK_TSG6 for use in medicine, and particularly for use in the treatment of osteoarthritic pain. Also described is a method of treatment of OA pain involving the administration of LINK_TSG6, and the use of LINK_TSG6 in the manufacture of a medicament for the treatment of osteoarthritic pain. The treatment may be for a subject in need thereof.

In preferred aspects LINK_TSG6 is administered subcutaneously or intra-articularly. Treatment may be once per week or less frequently, such as once per month. In some aspects, the treatment involves subcutaneous administration of 10 mg to 150 mg per month, 20 mg to 140 mg per month, 30 mg to 130 mg per month, 40 mg to 120 mg per month or 50 mg to 110 mg per month. In some aspects, the treatment involves subcutaneous administration of about 50 mg per month, about 60 mg per month, about 70 mg per month, about 80 mg per month, about 90 mg per month or about 100 mg per month of LINK_TSG6. In other aspects, the treatment involves intra-articular administration of 2 mg to 30 mg, 3 mg to 28 mg, 4 mg to 25 mg, 5 mg to 23 mg, 6 mg to 20 mg, or between 10 and 20 mg per month of LINK_TSG6.

In some aspects described herein, the treatment may encompass intra-articular administration of at least 3, least 5, at least 10, at least 20, at least 30, at least 40, at least 50, at least 60, at least 70, at least 80, at least 90, at least 100, at least 110, at least 120, at least 130, at least 140, at least 150, at least 160, at least 170 or at least 180 mg LINK_TSG6.

In some aspects, LINK_TSG6 is administered into an osteoarthritic joint. In some cases, it is administered distant to an osteoarthritic joint. In some cases, a patient or subject may have a plurality of osteoarthritic joints, and the LINK_TSG6 is administered into one of the osteoarthritic joints. In some cases, the LINK_TSG6 is administered into several of the osteoarthritic joints.

In some cases, the LINK_TSG6 is used to treat direct pain. That is, the LINK_TSG6 is used to treat pain in a joint resulting from osteoarthritis in that joint. The LINK_TSG6 may alternatively, or additionally, be used to treat referred pain. Referred pain is pain perceived at a location other than the site of the osteoarthritis.

In some aspects, LINK_TSG6 is administered once per 7 days, once per 10 days, once per 14 days, once per 21 days, once per 30 days, once per 45 days, once per 60 days, once per 90 days, or less than once per 90 days. The analgesic (pain relieving) effects may last for 5, 6, 7, 8, 9, 10, 11, 12, 13, 14, 15, 16, 17, 18, 19, 20, 21, 22, 23, 24, 25, 26, 27, 28, 29, 30, 31 or more days.

In some aspects disclosed herein, the LINK_TSG6 polypeptide comprises, consists, or consists essentially of the amino acid sequence of SEQ ID NO: 7 or SEQ ID NO: 9. In some aspects, the LINK_TSG6 polypeptide comprises, consists, or consists essentially of an amino acid sequence having at least 80% identity to the amino acid sequence of SEQ ID NO: 7 or SEQ ID NO: 9.

In a further aspect, the disclosure provides a composition comprising LINK_TSG6 that is formulated for subcutaneous administration. The composition may comprise at least 50 mg, at least 60 mg, at least 70 mg, at least 80 mg, at least 90 mg or at least 100 mg per dose of LINK_TSG6.

In a yet further aspect, the disclosure provides a composition comprising LINK_TSG6 that is formulated for intra-articular administration. The composition may comprise at least 1 mg/ml, at least 2 mg/ml, at least 3 mg/ml, at least 4 mg/ml, at least 5 mg/ml, at least 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml or more than 20 mg/ml of LINK_TSG6.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. Sequences relevant to the present disclosure.

FIG. 2. Link_TSG6 reduces direct OA joint pain. Link_TSG6 treatment by sub-cutaneous (s.c.) administration A. 1×/week (top; high 55 μg, low 13.75 μg per dose) or B. 1×/month (bottom; high 220 μg, low 55 μg per dose) dose-dependently reduces pain associated with knee-joint flexion in the mouse partial medial meniscectomy (pMx) model of OA, assessed by vocalisations. Significance was determined using a 1-way ANOVA with Tukey's post-hoc test: * p<0.05, ** p<0.01, *** p<0.001 compared to corresponding vehicle control.

FIG. 3. Link_TSG6 reduces thermal sensitivity. A. Link_TSG6 treatment by sub-cutaneous (s.c.) administration 1×/week reduces referred thermal sensitivity (assessed by paw withdrawal latencies to a cold (10° C.) stimulus) in the ipsilateral (surgically-operated) paw (top) using the mouse partial medial meniscectomy (pMx) model of OA. A significant analgesic effect is also apparent in the contralateral paw (bottom). B. Link_TSG6 treatment 1×/month s.c. reduces referred thermal sensitivity in the ipsilateral paw (top) of mouse pMx model of OA. A significant analgesic effect is also apparent in the contralateral paw (bottom). Significance was determined using a 1-way ANOVA with Tukey's post-hoc test: * p<0.05, ** p<0.01, *** p<0.001 compared to corresponding vehicle control.

FIG. 4. Link_TSG6 reduces mechanical sensitivity. A. Link_TSG6 treatment by sub-cutaneous (s.c.) administration 1×/week reduces referred mechanical sensitivity (measured by paw withdrawal threshold) in the ipsilateral (surgically-operated) paw (top) using the mouse partial medial meniscectomy (pMx) model of OA. A significant analgesic effect is also apparent in the contralateral paw (bottom). B. Link_TSG6 treatment 1×/month reduces referred mechanical sensitivity in the ipsilateral paw (top) using the mouse pMx model of OA. A significant analgesic effect is also apparent in the contralateral paw (bottom). Significance was determined using a 1-way ANOVA with Tukey's post-hoc test: * p<0.05, ** p<0.01, *** p<0.001 compared to corresponding vehicle control.

FIG. 5. Link_TSG6 improves weight bearing. A. Link_TSG6 treatment (1×/week) dose-dependently improves weight bearing (measured by dual channel weight averaging) in the mouse pMx model of OA when using either sub-cutaneous (s.c.) (top) or intra-articular (i.a.) (bottom) administration. B. Link_TSG6 treatment (1×/month) dose-dependently improves weight bearing in the mouse pMx model of OA when using either sub-cutaneous (s.c.) (top) or intra-articular (i.a.) (bottom) administration. Significance was determined using a 1-way ANOVA with Tukey's post-hoc test: * p<0.05, ** p<0.01, *** p<0.001 compared to corresponding vehicle control.

FIG. 6. Link_TSG6 reduces tactile allodynia in the ACLTpMMx rat. OA was induced in the right knees of male Lewis rats by ACLT and pMMx (30%). Link_TSG6 treatment (40 □g) or vehicle was administered i.a. at 7, 14 and 21 days post-surgery. After 28 days tactile allodynia was assessed by determining withdrawal thresholds to von Frey hairs applied to hind paws of the (A) un-operated control legs and (B) operated legs. Mean values ±SD indicated by horizontal bars (n=15). Data were analysed by one-way ANOVA with Tukey's post hoc test; *p<0.05, ***p<0.001, ****p<0.0001, relative to vehicle control.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### TSG-6 (Tumor Necrosis Factor-Stimulated Gene-6)

TSG-6 is a secreted protein composed of two modular domains. TSG-6 is not usually constitutively expressed in adult tissues, rather being induced in response to inflammatory mediators. During inflammation, TSG-6 is an endogenous protector of tissues. Many of the immunomodulatory and tissue-protective effects of by mesenchymal stem/stromal cells (MSCs) are mediated by their secretion of TSG-6.

Recombinant full-length TSG-6 protein has been shown to have anti-inflammatory and tissue protective effects in a wide range of disease models, such as atherosclerosis, myocardial infarction, hypertrophic scarring, colitis, autoimmune diabetes, rheumatoid arthritis, traumatic brain injury or acute lung injury.

Full length TSG-6 is hard to make, insoluble and prone to aggregation. As disclosed herein, these disadvantages are not associated with LINK_TSG6, a short recombinant peptide comprising the LINK module of human TSG-6. This short polypeptide is easier to make than full length TSG-6, and is highly soluble and stable in solution.

While constitutively expressed in a few tissues, TSG-6 is generally upregulated wherever there is inflammation. For the most part TSG-6 exhibits anti-inflammatory and tissue protective properties, but has been implicated as sometimes playing a role in disease pathology, for example, in the lung. While being made by a broad range of cell types, it was the finding that TSG-6 is produced by mesenchymal stem/stromal cells (MSCs) in response to inflammatory signals, and that it mediates many of their immunomodulatory and reparative activities, which has led to a wealth of publications on the therapeutic effects of this intriguing molecule across a wide range of disease models.

TSG-6 is a relatively small protein, with a molecular mass of only ˜35-38 kDa, being mainly composed of two modular domains (Link and CUB_C). Given TSG-6's size, it has a surprisingly large number of activities, including the modulation of immune and stromal cell function and its contribution to extracellular matrix formation, mechanics and remodelling. It is the ability of TSG-6 to regulate matrix organization, and to control the association of matrix molecules with cell surface receptors and with extracellular signalling factors (e.g. chemokines), that likely underlies its diverse functional repertoire. In this regard, TSG-6 interacts with a large array of ligands, such as glycosaminoglycans (GAGs), proteoglycan (PG) core proteins and other matrix components, and binds directly to multiple chemokines and bone morphogenetic proteins (BMPs). One particularly unusual function of TSG-6 is its role as an enzyme that catalyses the covalent modification of the non-sulfated GAG hyaluronan (HA) with so-called heavy chains (HCs) from the inter-α-inhibitor (IαI) family of proteoglycans. This process, mediated by the full-length TSG-6 protein, but not LINK_TSG6 polypeptides containing only a fragment of TSG-6, results in the formation of HC·HA complexes, and is essential for mammalian ovulation and fertilisation, and also occurs in many other contexts (e.g. inflammation) where HC·HAs either confer tissue protection or contribute to pathological processes.

The sites and contexts of TSG-6 expression, its structure and ligand-binding properties, and how these together underpin its diverse biology and therapeutic potential at a molecular level are reviewed in Day & Milner (Matrix Biology (2019) 78-79, 60-83).

LINK_TSG6 polypeptide as disclosed herein comprises the Link module of human or mammalian TSG-6. In some embodiments, the TSG-6 polypeptide comprises or consists essentially of the amino acid sequence according to SEQ ID NO: 2 or SEQ ID NO: 5. The Link module corresponds to residues 37-128 of SEQ ID NO:s 2 and 5, and is shown in SEQ ID NO: 7.

The Link module is responsible for the hyaluronan (HA) binding activity, chondroitin sulfate binding activity, aggrecan binding activity, inter-α-inhibitor (IαI) binding activity, heavy chain (HC) 1 binding activity, HC2 binding activity, HC3 binding activity, bikunin binding activity, versican binding activity, dermatan sulfate binding activity, pentraxin-3 binding activity, thrombospondin-1 binding activity, thrombospondin-2 binding activity, fibronectin binding activity, heparin/heparan sulfate binding activity, RANKL binding activity of TSG-6, bone morphogenetic protein (BMP)-2 binding activity, BMP-4 binding activity, BMP-5 binding activity, BMP-6 binding activity, BMP-7 binding activity, BMP-13 binding activity, BMP-14 binding activity, CXCL4 binding activity, CXCL6 binding activity, CXCL8 binding activity, CXCL11 binding activity, CXCL12 binding activity, CCL2 binding activity, CCL5 binding activity, CCL7 binding activity, CCL19 binding activity, CCL21 binding activity or CCL27 binding activity.

LINK_TSG6 may be a fragment of TSG-6 exhibiting one or more of hyaluronan (HA) binding activity, chondroitin sulfate binding activity, aggrecan binding activity, inter-α-inhibitor (IαI) binding activity, heavy chain (HC) 1 binding activity, HC2 binding activity, HC3 binding activity, bikunin binding activity, versican binding activity, dermatan sulfate binding activity, pentraxin-3 binding activity, thrombospondin-1 binding activity, thrombospondin-2 binding activity, fibronectin binding activity, heparin/heparan sulfate binding activity, RANKL binding activity, bone morphogenetic protein (BMP)-2 binding activity, BMP-4 binding activity, BMP-5 binding activity, BMP-6 binding activity, BMP-7 binding activity, BMP-13 binding activity, BMP-14 binding activity, CXCL4 binding activity, CXCL6 binding activity, CXCL8 binding activity, CXCL11 binding activity, CXCL12 binding activity, CCL2 binding activity, CCL5 binding activity, CCL7 binding activity, CCL19 binding activity, CCL21 binding activity or CCL27 binding activity.

US2015/0057229 describes the use of LINK_TSG6 in inhibiting cartilage degradation.

The LINK domain of TSG-6 (LINK_TSG6) may be the region of full-length TSG-6 N-terminal to the CUB_C domain. As such, the LINK_TSG6 protein may lack all or part of the CUB_C domain. In preferred aspects, the LINK_TSG6 polypeptide may comprise the N-terminal region of the CUB_C domain.

The LINK domain may contain the amino acid sequence of SEQ ID NO: 7 or SEQ ID NO: 9. LINK_TSG6 polypeptide comprises, consists, or consists essentially of (i) the amino acid sequence of SEQ ID NO: 7 or SEQ ID NO: 9, or (ii) an amino acid sequence having at least 80% identity to the amino acid sequence of SEQ ID NO: 7 or 9.

LINK_TSG6 is preferably a polypeptide comprising or consisting of: (i) the amino acid sequence of SEQ ID NO: 7 or 9, or (ii) an amino acid sequence having one of at least 80%, 85%, 90%, 91%, 92%, 93%, 94%, 95%, 96%, 97%, 98%, 99% or 100% identity to the amino acid sequence of SEQ ID NO: 7 or 9.

In some cases, LINK_TSG6 consists of the amino acid sequence of SEQ ID NO:9 and optionally 1, 2, 3, 4, 5, 6, 7, 8, 9, 10, 11, 12, 13, 14, 15, 16, 17, 18, 19 or 20 additional amino acids at the C-terminus, the N-terminus, or at each of the C-terminus and the N-terminus. In some cases, LINK_TSG6 consists of the amino acid sequence of SEQ ID NO:7 and optionally 1, 2, 3, 4, 5, 6, 7, 8, 9, 10, 11, 12, 13, 14, 15, 16, 17, 18, 19 or 20 additional amino acids at the C-terminus, the N-terminus, or at each of the C-terminus and the N-terminus.

Accordingly, the LINK_TSG6 polypeptide may comprise:
(a) the amino acid sequence of SEQ ID NO: 7;
(b) a variant thereof having at least 50% identity to the amino acid sequence of SEQ ID NO: 7 and having RANKL binding activity; or
(c) a fragment of either (a) or (b) having CXCL4 binding activity, CXCL6 binding activity, CXCL8 binding activity, CXCL11 binding activity, CXCL12 binding activity, CCL2 binding activity, CCL5 binding activity, CCL7 binding activity, CCL19 binding activity, CCL21 binding activity or CCL27 binding activity.

The LINK_TSG6 polypeptide may consists of, or consist essentially of, the sequence shown in SEQ ID NO: 7.

SEQ ID NO: 9 shows a recombinant polypeptide which includes the Link module of TSG-6 (LINK_TSG6).

Accordingly, the TSG-6 polypeptide used in the invention may preferably comprises:
(a) the amino acid sequence of SEQ ID NO: 9;
(b) a variant thereof having at least 50% identity to the amino acid sequence of SEQ ID NO: 9 and having RANKL binding activity; or
(c) a fragment of either (a) or (b) having CXCL4 binding activity, CXCL6 binding activity, CXCL8 binding activity, CXCL11 binding activity, CXCL12 binding activity, CCL2 binding activity, CCL5 binding activity, CCL7 binding activity, CCL19 binding activity, CCL21 binding activity or CCL27 binding activity.

The LINK_TSG6 polypeptide preferably consists of, or consists essentially of, the sequence shown in SEQ ID NO: 9.

Amino acid identity may be calculated using any suitable algorithm. For example the UWGCG Package provides the BESTFIT program which can be used to calculate homology (for example used on its default settings) (Devereux et al. (1984) Nucleic Acids Research 12, 387-395). The PILEUP and BLAST algorithms can be used to calculate homology or line up sequences (such as identifying equivalent or corresponding sequences (typically on their default settings), for example as described in Altschul (1993) J. Mol. Evol. 36, 290-300; Altschul et al. (1990) J. Mol. Biol. 215, 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Nat. Acad. Sci. USA 89, 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90, 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The variant sequences typically differ by at least 1, 2, 5, 10, 20, 30, 50 or more mutations (which can be substitutions, deletions or insertions of amino acids). For example, from 1 to 50, 2 to 30, 3 to 20 or 5 to 10 amino acid substitutions, deletions or insertions can be made. The modified polypeptide may generally retain CXCL4 binding activity, CXCL6 binding activity, CXCL8 binding activity, CXCL11 binding activity, CXCL12 binding activity, CCL2 binding activity, CCL5 binding activity, CCL7 binding activity, CCL19 binding activity, CCL21 binding activity or CCL27 binding activity, preferably in a dose-dependent manner. The substitutions are preferably conservative substitutions, for example according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

A LINK_TSG6 polypeptide used in the invention is typically at least 10, for example at least 15, 20, 25, 30, 40, 50, 60, 70, 80, 90 or more amino acids in length, up to 100, 150, 200 or 250 amino acids in length, as long as it retains the CXCL4 binding activity, CXCL6 binding activity, CXCL8 binding activity, CXCL11 binding activity, CXCL12 binding activity, CCL2 binding activity, CCL5 binding activity, CCL7 binding activity, CCL19 binding activity, CCL21 binding activity or CCL27 binding activity of TSG-6. Preferably, the polypeptide includes the sequence shown in SEQ ID NO: 7. Fragments of the amino acid sequence of SEQ ID NO: 2, SEQ ID NO: 3, SEQ ID NO: 5, SEQ ID NO: 6, SEQ ID NO: 7 or SEQ ID NO: 9 preferably contain the residues shown to be essential for hyaluronan binding in Mahoney et al. (2001) J. Biol. Chem. 276, 22764-22771 and Blundell et al. (2003) J. Biol. Chem. 278, 49261-49270. Fragments of the amino acid sequence of SEQ ID NO: 2 or 5 preferably contain the residues Lys-46 and/or Tyr-47 and/or Tyr-94 and/or Phe-105 and/or Tyr-113 of SEQ ID NO: 2 or 5. Most preferably, the fragment of SEQ ID NO: 2 or 5 contains each of residues Lys-46, Tyr-47, Tyr-94, Phe-105 and Tyr-113 of SEQ ID NO: 2 or 5.

Fragments of the amino acid sequence of SEQ ID NO: 7 may be used in the invention. Such fragments preferably contain the residues Lys-10 and/or Tyr-11 and/or Tyr-58 and/or Phe-69 and/or Tyr-77 of SEQ ID NO: 7. Most preferably, the fragment of SEQ ID NO: 7 contains each of residues Lys-10, Tyr-11, Tyr-58, Phe-69 and Tyr-77 of SEQ ID NO: 7.

Fragments of the amino acid sequence of SEQ ID NO: 9 preferably contain the residues Lys-11 and/or Tyr-12 and/or Tyr-59 and/or Phe-70 and/or Tyr-78 of SEQ ID NO: 9. Most preferably, the fragment of SEQ ID NO: 9 contains each of residues Lys-11, Tyr-12, Tyr-59, Phe-70 and Tyr-78 of SEQ ID NO: 9. The TSG-6 polypeptides used in the invention may be chemically modified, e.g. post-translationally modified. For example, they may be glycosylated, phosphorylated or comprise modified amino acid residues. They may be modified by the addition of histidine residues to assist their purification or by the addition of a transmembrane sequence to promote insertion into the cell membrane. Such modified polypeptides fall within the scope of the term “polypeptide” used herein.

Suitable assays for determining the ability of a TSG-6 polypeptide to bind to HA, chondroitin sulfate, aggrecan, inter-α-inhibitor (IαI), heavy chain (HC) 1, HC2, HC3, bikunin, versican, dermatan sulfate, pentraxin-3, thrombospondin-1, thrombospondin-2, heparin/heparan sulfate, fibronectin, RANKL bone morphogenetic protein (BMP)-2, BMP-4, BMP-5, BMP-6, BMP-7, BMP-13, BMP-14, CXCL4, CXCL6, CXCL8, CXCL11, CXCL12, CCL2, CCL5, CCL7, CCL19, CCL21 and CCL27 are well-known in the art (Getting et al. (2002) J. Biol. Chem. 277, 51068-51076; Mahoney et al. (2005) J. Biol. Chem. 280, 27044-27055; Salustri et al. (2004) Development 131, 1577-1586; Parkar et al. (1997) FEBS Lett. 410, 413-417; Parkar et al. (1998) FEBS Lett. 428, 171-176; Mahoney et al. (2001) J. Biol. Chem. 276, 22764-22771; Nentwich et al. (2002) J. Biol Chem. 211, 15354-15362; Kuznetsova et al. (2005) J. Biol. Chem. 280, 30899-30908; Dyer et al. (2014) J. Immunol 192, 2177-2185; Dyer et al. (2016) J. Biol. Chem. 291, 12627-12640; Mahoney et al. (2008) J. Biol. Chem 283, 25952-25962; Marson et al. (2009) Glycobiology 19, 1537-1546; Kuznetsova et al. (2006) J. Cell Sci. 119, 4499-4509; Kuznetsova et al. (2008) Matrix Biology 27, 201-210); and Day & Milner. (2019) Matrix Biology. 78-79, 60-83.

TSG-6 polypeptides for use in the invention may be in a substantially isolated form. It will be understood that the polypeptide may be mixed with carriers or diluents which will not interfere with the intended purpose of the polypeptide and still be regarded as substantially isolated. A polypeptide for use in the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention.

LINK_TSG6 polypeptides for use in the present invention may be natural or non-naturally occurring polypeptides. Polypeptides may be isolated from any suitable organism that expresses a TSG-6 polypeptide. The TSG-6 polypeptide may be isolated from a human or another suitable mammal, such as primates, rats or mice. Alternatively, TSG-6 polypeptide may be isolated from a fish or an amphibian. Polypeptides for use in the invention may also be prepared as fragments of such isolated polypeptides.

Further, the LINK_TSG6 polypeptides may also be made synthetically or by recombinant means. For example, a recombinant LINK_TSG6 polypeptide may be produced by transfecting cells in culture with an expression vector comprising a nucleotide sequence encoding the polypeptide operably linked to suitable control sequences, culturing the cells, extracting and purifying the LINK_TSG6 polypeptide produced by the cells. Methods for the recombinant production of polypeptides are well-known in the art (for example, Sambrook et al., 2001, Molecular Cloning: a laboratory manual, 3^{rd} edition, Cold Harbour Laboratory Press). Preferably, the LINK_TSG6 polypeptide is made in a bacteria, such as E. coli.

The amino acid sequence of LINK_TSG6 polypeptides for use in the invention may be modified to include non-naturally occurring amino acids or to increase the stability of the compound. When the polypeptides are produced by synthetic means, such amino acids may be introduced during production. The polypeptides may also be modified following either synthetic or recombinant production.

In some aspects, LINK_TSG6 polypeptides used herein are not comprised within a fusion protein. That is, LINK_TSG6 is not fused to a further peptide or polypeptide. In some cases, LINK_TSG6 is not a fusion protein with a growth factor, such as IGF-1.

LINK_TSG6 polypeptides for use in the invention may also be produced using D-amino acids. In such cases the amino acids will be linked in reverse sequence in the C to N orientation. This is conventional in the art for producing such polypeptides.

A number of side chain modifications are known in the art and may be made to the side chains of the LINK_TSG6 polypeptides, provided that the polypeptides retain ability to reduce osteoarthritic pain.

### Osteoarthritis

The present disclosure relates to the treatment of osteoarthritic pain. Osteoarthritic pain is pain associated with osteoarthritis. The osteoarthritic pain may be caused by osteoarthritis. In other words, osteoarthritis may cause a patient's joints to become painful and stiff.

Osteoarthritis (OA) is the most common form of arthritis worldwide. It is a long-term chronic disease caused by the deterioration of cartilage in joints and is characterised by pain and a loss of mobility in the affected joints. Almost any joint can be affected by osteoarthritis, but the condition most often causes problems in the knees, hips and small joints of the hands and wrists. For example, joints of the hands and wrists might be the joints of the fingers and thumb (interphalangeal joints), the joint at the base of the finger or thumb (metacarpophalangeal joint), the base of the hands (carpometacarpal) or the wrist (scaphotrapeziotrapezoidal, intercarpal and midcarpal/radiocarpal joints). Other joints that may be affected include the joints of the spine, shoulders, elbows, joints of the ankles (including the talocrural joint and the subtalar joint, and the tibiofibular joint) and small joints of the feet (for example metatarsophalangeal joint, proximal interphalangeal joint and distal phalangeal joint).

OA may be diagnosed based on a description of the symptoms including location and patterns of any pain experienced. This can include a history of joint pain that is worsened by movement, or time of day. Description of symptoms can also include stiffness, particularly after rising in the morning, and experiencing limited joint movement. Diagnosis may also take into account any family history of OA.

OA is commonly diagnosed by a physical examination to check for symptoms such as joint tenderness, stiffness and restricted movement of the joint, altered gait, clicking noises or crepitus when moving the joint, bony swellings or misshapen joints such as Heberden's and Bouchard's nodes, joint instability, excess fluid or weakness of muscles supporting the joint.

In some cases, OA diagnosis may include using X-rays of affected joints, to identify symptoms that include narrowing of the joint space between adjacent bones, calcium deposits in the joint and bone spur formation (growths that develop where bones meet in the joint). In some cases, magnetic resonance imaging (MRI) may also be used in OA diagnosis. MRI can image soft tissues including cartilage and bone marrow and may show bone marrow lesions and cartilage breakdown.

OA severity is often characterised by ‘stage’. The 4 Stages of OA progression are 1—minor, 2—mild, 3—moderate or 4—severe. Stage 0 represents ‘normal’ knee health (no signs of joint damage or known impairment). OA does not develop at a uniform rate. OA can worsen over weeks, months, years or decades. Progression of OA depends on many factors including age, genetics, overall health and activity levels, weight/BMI and how much the affected joints are used. In many cases, the level of pain is linked to structural changes in the joint. However, some patients experience chronic pain without substantial structural changes, such as cartilage loss.

Patients with stage 1 (minor) OA will develop minor structural changes and typically experience little or no pain. At stage 1, minor growth of bone spurs may occur in affected joints.

Patients with stage 2 (mild) OA may experience stiffness, discomfort and in some cases may experience pain in the affected joints. Stiffness and discomfort may be exacerbated by long periods of sitting, in the morning, or after a workout. Noticeable bone spurs may be detected by X-rays and cartilage may start to break down. Stage 2 OA may require treatment with pain relief or other treatment such as exercise regimes, weight loss or wearing joint supports or braces.

Stage 3 (moderate) OA occurs when there is obvious erosion of the cartilage in the affected joint. The gap between bones in the joint may be narrowed and bone spurs may develop or continue to develop between bones in the joint. Joints may look misshapen or crooked. The joint may be inflamed and patients with stage 3 OA may experience frequent pain and discomfort during daily activities such as walking, running, kneeling or extending the affected joint. Stiffness and discomfort may be increased after sitting for extended periods or in the morning. Patients are likely to seek pain relief.

Patients with stage 4 (severe) OA experience the most advanced form of OA. Stage 4 may be characterised by little to no cartilage and considerably reduced joint space between the bones. There may be decreased synovial fluid in the joint and increased friction, pain and discomfort when moving the affected joint. In some cases, however, synovial fluid in the joint is increased due to oedema. Bone spur growths are likely to have multiplied. Joints may look misshapen or crooked. Typically pain at stage 4 OA is greater and can be described as chronic pain, that is severe and excruciating, stage 4 OA pain can also make everyday activities such as walking and taking stairs a challenge.

### Osteoarthritic Pain

OA pain is pain associated with OA. OA pain may be framed in a biopsychological framework, with biological, psychological and social factors all contributing. Chemical mediators are released into the inflamed joint, sensitizing primary afferent nerves, such that normally innocuous movements (such as increased physical activity, high heeled shoes or weather changes) now elicit a painful response. This is the neurophysiological basis of allodynia (the sensation of pain in response to a normally non-painful stimulus). Overtime, increased neuronal activity can cause plasticity changes in the central nervous system, by a process known as “wind-up”, during which second order neurones in the spinal cord increase their firing rate, such that transmission of pain information to the somatosensory cortex is enhanced.

This central sensitization phenomenon intensifies pain sensation and can even lead to pain response from regions of the body remote from the inflamed joint (i.e. referred pain).

The structural determinants of OA pain are likely to involve multiple pathways. OA pain is unlikely to be directly generated from damaged cartilage since joint cartilage is a neural (has no nervous tissue) and is avascular (lacks blood vessels). Instead, other symptom sources in the joint include subchondral bone, periosteum, periarticular ligaments, periarticular muscle spasm, synovium and joint capsule, which are all richly supplied with nerves and are a source of nociception in OA. OA pain may be related to structural causes such as bone marrow lesions, sub-articular bone attrition, synovitis (inflammation of the synovial membrane) and effusion (excess synovial fluid accumulates in or around the knee joint). Such structural causes may be detected using MRI. Previous work by Lo et al. has found a strong association of bone marrow lesions and effusion with pain in osteoarthritis (Lo, et al. Arthritis & Rheumatism. 2008; 56(9):S790. Abstract).

In some cases, OA joint pain may be due to plasticity of the nerves. For example, tissues that are usually a neural such as cartilage may undergo innervation in patients with OA, meaning that cartilage which is usually insensitive may become a source of pain.

Constitutional factors that can predispose to symptoms include self-efficacy, pain catastrophizing and the social context of arthritis (e.g. social support, pain communication), all of which are important considerations in understanding the pain experience.

OA pain may be described as achy, and may be worse after moving the affected joint. In some cases, OA pain is worse at the end of the day. OA pain may increase or lessen after eating certain foods. OA pain is not always relative to OA stage, in some cases patients may experience severe pain from a small amount of joint damage. Pain from moderate and severe OA can have a severe impact on quality of life. OA pain can result in sleep disturbances that can lead to fatigue and depression. In some cases, OA pain that varies from day to day may cause more sleep disturbances as it is harder to predict and to adjust to the pain. It may be that poor sleep increases sensitivity to OA pain. In some cases, patients with OA may find it beneficial to attend a support group for those with chronic pain.

Osteoarthritic pain usually gets worse over time however in some cases it can come and go, and does not always get worse in a linear fashion. Periods of moderate to severe pain may be followed by periods of improvement. Sometimes OA pain may be worse at different times of year, or be weather-sensitive. This may be due to influence of barometric pressure and ambient temperature (McAlindon et al., 2007. Am J Med. 120(5):429-434). In some cases, osteoarthritic pain is worse in autumn and winter, or during periods of greater barometric pressure or colder ambient temperature are associated with greater pain. In some cases, applying hot or cold packs to the affected area can temporarily reduce OA pain.

Relief of OA pain may also be important for surgical outcomes after joint replacement. For example, pain outcomes of knee arthroplasty (knee replacement surgery) can be affected by preoperative pain perception. In some cases, preoperative pain catastrophizing (the belief that pain will get worse and that one is helpless to deal with it) is a predictor of poor post-surgical pain outcome (Riddle et al., 2010, Clin Orthop Relat Res. 468(3):798-806).

Osteoarthritic pain is different to neuropathic pain. Neuropathic pain is pain caused by damage or disease affecting the somatosensory nervous system, and may be associated with abnormal sensations called dysesthesia or pain from normally non-painful stimuli (allodynia). It may have continuous and/or episodic (paroxysmal) components. In contrast, osteoarthritic pain is a nociceptive pain. Nociceptive pain is pain reported to the brain by the nervous system, rather than originating in the nervous system itself.

In certain aspects disclosed herein, osteoarthritic pain is not neuropathic pain, or is not osteoarthritic pain with a neuropathic component. In certain aspects disclosed herein, the patient does not have neuropathic pain. In some cases, the OA pain is, or includes, a referred pain. The referred pain may be pain in an area of the body that is distant to an osteoarthritic joint.

OA pain may be measured using the Western Ontario and McMaster Universities Osteoarthritis Index (WOMAC) and the Medical Outcomes Study Short-Form 36 (SF-36) scales.

WOMAC is a questionnaire that subdivides OA pain into 5 aspects; during walking, using stairs, in bed, sitting or lying, and standing upright. In addition to pain, WOMAC also includes questions on 2 aspects of stiffness; after first waking and later in the day, and assessment of 17 aspects of ‘physical function’ when completing activities such as descending stairs, ascending stairs, rising from sitting, standing, bending, walking, getting in or out of a car, shopping, putting on socks, taking off socks, rising from bed, lying in bed, getting in or out of the bath, sitting, getting on and off the toilet, heavy domestic duties and light domestic duties. The test questions are scored on a scale of 0-4, which correspond to: None (0), Mild (1), Moderate (2), Severe (3), and Extreme (4). The scores for each subscale are summed up, with a possible score range of 0-20 for Pain, 0-8 for Stiffness, and 0-68 for Physical Function. Usually a sum of the scores for all three subscales gives a total WOMAC score out of 96, where a higher score is indicative of pain, stiffness and limited physical function. A reduction in WOMAC pain score, or a reduction in total WOMAC score may indicate that treatment of OA pain has been successful.

SF-36 includes questions on how much bodily pain a patient has had during the past 4 weeks (on a scale of 1-6, 1—none, 2—very mild, 3—mild, 4—moderate, 5—severe, 6—very severe), and how much pain has interfered with normal work (including both work outside the home and housework), where 1—not at all, 2—a little, 3—moderately, 4—quite a bit and 5—extremely. The questions may also include whether the patients' health is limiting them from activities such as such as running, lifting objects, participating in sports, pushing a vacuum cleaner, climbing stairs, bending, kneeling, or stooping, walking various distances or bathing or dressing yourself (for each activity; 1—limited a lot, 2—limited a little or 3—not limited at all).

The qualitative aspects of OA pain may be assessed using a scale such as the Short-Form McGill Pain Questionnaire (MPQ-SF). The MPQ-SF includes a ‘sensory subscale’ and an ‘affective subscale’. Sensory subscale descriptors may include for example: throbbing, shooting, stabbing, sharp, cramping, gnawing, hot-burning, aching, heavy, tender, splitting. Affective subscale may include descriptors such as ‘tiring-exhausting’, sickening, fearful and ‘punishing-cruel’. Each descriptor or aspect of pain is scored using the scale of 0—none, 1—mild, 2—moderate or 3—severe.

In some cases it may be that OA pain can be measured using questionnaires such as the Measure of Intermittent and Constant Osteoarthritis Pain (ICOAP) (https://www.oarsi.org/research/outcome-measures). ICOAP measures constant OA pain separately to “pain that comes and goes”. Constant OA pain may be measured by questions that may include how intense the pain has been, how much pain has affected sleep, how much pain has impacted overall quality of life, how much pain has caused frustration or annoyance, or upset and worry. Each aspect is scored as 0—not at all, 1—mildly, 2—moderately, 3—severely or 4—extremely. Infrequent OA pain or OA “pain that comes and goes” may also be measured by intensity, frequency (0—never, 1—rarely, 2—sometimes, 3—often, 4—very often), effect on sleep, effect on quality of life, how much OA pain has caused frustration or annoyance, or upset and worry. Each aspect except frequency is scored as 0—not at all, 1—mildly, 2—moderately, 3—severely or 4—extremely. These variables may be measured for example, over the past few days, or the past week. In some cases, they may be measured over a period of longer than a week.

### Patient

The methods disclosed herein may comprise a step of evaluating a subject or patient for evidence of OA and osteoarthritic pain. The patient may have osteoarthritis. In other words, the patient may have previously received a diagnosis of osteoarthritis. The patient may have previously been treated for osteoarthritic pain, for example, the patient may have previously received acetaminophen (paracetamol), an NSAID or an opiate/opioid analgesic to treat osteoarthritic pain. In some cases, the patient does not suffer from other types of pain. For example, the patient does not have or suffer from neuropathic pain.

The patient may be male or female. The patient may be of any age. For example, the patient may be at least 20, 25, 30, 35, 40, 45, 50, 55, 60, 65, 70, 75, 80, 85, 90, 95, 100 or more years old.

The subject to be treated may be any animal or human. The subject is preferably mammalian, more preferably human. The subject may be a non-human mammal, such as a dog, cat, horse or cow. The subject may be male or female. In some cases, osteoarthritic pain in a non-human mammal may be characterised by behavioural changes, such as walking with a limp, biting or snapping, vocalizing when handled or a decrease in play. The subject may be a patient. Therapeutic uses may be in human or animals (veterinary use). The subject or patient may be a subject or patient in need of treatment of OA pain. The subject or patient has, or is suspected of having, osteoarthritis. The subject or patient has pain that is, or is suspected to be, osteoarthritic pain. In other words, pain experienced by the patient or subject is caused by, or suspected to be caused by, osteoarthritis in the subject/patient. The subject/patient may have one or more osteoarthritic joints (i.e. a connection made between two or more bones that has one or more symptoms of osteoarthritis). The subject or patient may have a clinical diagnosis of osteoarthritis. The subject/patient may have stage I, stage II, stage III or stage IV osteoarthritis. Most likely, the subject/patient has stage II, stage III or stage IV osteoarthritis. The subject or patient may be self-diagnosed as having osteoarthritis.

### Treatments

Disclosed herein are methods of treating or preventing osteoarthritic pain. Treating osteoarthritic pain may result in, or aim to result in, the reduction or elimination of OA pain. The methods may involve reduction in one or more of a severity of pain, frequency of pain, effect of OA pain on sleep, effect of pain on quality of life, extent to which pain has caused frustration or annoyance, worry or upset, a reduction in pain when walking, standing, sitting, using stairs, performing domestic duties, dressing or bathing. Treatment may result in complete or partial resolution of OA pain. The treatment may result in the partial or complete alleviation of pain symptoms, such as the partial or complete alleviation of symptoms for at least one day following administration, at least two days following administration, at least three days following administration, at least four days following administration, at least five days following administration, at least six days following administration, at least seven days following administration, more than one week following administration, more than 10 days following administration, more than two weeks following administration, more than three weeks following administration, or one month or more following administration. The partial or complete alleviation of symptoms may occur for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days following administration. Preferably, the partial or complete alleviation of symptoms occurs for at least one week after administration.

Following treatment, the patient may have a reduction in WOMAC pain score in one or more of the 5 aspects, reduction in WOMAC pain score in 2 or more, 3 or more, 4 or more or all 5 aspects of the WOMAC pain score. In some cases, the reduction in score of one or more aspect may be by more than one point, or more than 2, 3 or 4 points. For example, the treatment may result in a reduction in at least one aspect of the WOMAC score by at least one point, or in at least two aspects of the WOMAC score by at least one point in each aspect, or in at least three aspects of the WOMAC score by at least one point in each aspect, or in at least 4 aspects of the WOMAC score by at least one point in each aspect, or in all 5 aspects of the WOMAC score by at least one point in each aspect. The treatment may result in a reduction in at least one aspect of the WOMAC score by at least two points, or in at least two aspects of the WOMAC score by at least two points in one aspect, or in at least three aspects of the WOMAC score by at least two points in one aspect, or in at least 4 aspects of the WOMAC score by at least two points in one aspect, or in all 5 aspects of the WOMAC score by at least two points in one aspect.

The treatment may result in a reduction in the WOMAC pain score to 0.

The patient may have a reduction in overall WOMAC score out of 96, for example a reduction by at least 5, 10, 15, 20, 25, 30, 35, 40, 45, 50, 55, 60, 65, 70, 75, 80, 90, 95 or complete reduction of WOMAC total score to 0.

In some cases, following treatment the patient may have a reduction by one or more points in SF-36 pain score, such as a reduction in score for how much pain they have experienced in the past 4 weeks, or since treatment. This may be a reduction by 1, 2, 3, 4 or 5 points. The patient may also experience a reduction on the SF-36 scale of how much the pain has interfered with normal work, by one or more points. In some cases, the reduction may be by 2, 3 or 4 points. It may be that a reduction by one or more points is recorded for both aspects of pain (i.e. how much pain the patient experiences and how much this pain has interfered with normal work).

Following treatment, the patient may experience improvement in the qualitative aspects of OA pain as assessed using MPQ-SF. The improvement may be quantified as a reduction in score in one or more of the 11 aspects of the ‘sensory subscale’. The improvement may be in 1, 2, 3, 4, 5, 6, 7, 8, 9, 10 or all 11 aspects of the sensory subscale. In some cases, the reduction in score of one or more aspect may be by one or more points, 2 or more points or by 3 points. In some cases, the improvement may be a reduction in score in one or more of the 4 aspects of the ‘affective subscale’. The improvement may be in 1, 2, 3 or all 4 aspects of the sensory subscale. It may be that there is an improvement in one or more aspects of both the sensory and affective subscales.

Following treatment, a patient may experience an improvement in intermittent and/or constant pain as measured using ICOAP. The improvement may be quantified as a reduction in score in one or more of the aspects of constant pain. In some cases, the improvement be a reduction in two or more aspects of constant pain, in 3 or more, 4 or more, or a reduction in score across all aspects of constant pain. It may be that a reduction in score is by more than one point, such as by 2, 3 or 4 points. It may be that one or more aspects, or total constant pain score may be reduced to 0. Patients may also experience an improvement in intermittent pain, such as a reduction in frequency or intensity score. In some cases, the improvement may be a reduction in two or more aspects of intermittent pain, in 3 or more, 4 or more, 5 or more, or a reduction in score across all aspects of intermittent pain. It may be that a reduction in score is by more than one point, such as by 2, 3 or 4 points. It may be that one or more aspects, or total intermittent pain score may be reduced to 0.

It may be that following treatment, the patient has a reduced pain score using another, clinically appropriate measure of pain intensity, frequency, or effect on day to day activities and behaviours, or mental health.

### Routes of Administration

Methods described herein may involve the administration of LINK_TSG6 by any route, such as intra-articular, topical, parenteral, systemic, intravenous, intra-arterial, intramuscular, intrathecal, intraocular, subcutaneous, oral or transdermal routes of administration which may include injection.

In preferred aspects, the treatment involves subcutaneous or intra-articular administration.

In some cases, the treatment involves the administration of LINK_TSG6 to a site distant to the site of osteoarthritic pain. A distant site may be any suitable site that is located away from the site of osteoarthritic pain. The distant site is usually a site suitable for subcutaneous administration. In some cases, LINK_TSG6 may be administered subcutaneously to the abdomen, arm, thigh, buttock or hip. For example, the upper outer area of the arm, front and outer sides of the thigh, the upper outer area of the buttock or the upper hip. The distant site may be a site in the same limb as the site of osteoarthritic pain, such as near the opposite end of a bone to an osteoarthritic joint. For example, where the site or osteoarthritic pain is a knee joint with osteoarthritis, the LINK_TSG6 may be administered near to the top of the femur, thigh or hip in the same leg as the osteoarthritic knee. In some cases, the distant site is a contralateral site (i.e. a site on the other side of the body to the site of osteoarthritic pain), such as the leg opposite the leg comprising the knee with osteoarthritic pain. In some cases the distant site is a different limb to the site of osteoarthritic pain, such as an arm, or the abdomen.

In some cases, the treatment involves intra-articular (i.a.) administration of LINK_TSG6. The i.a. administration may involve administration to an osteoarthritic joint. The i.a. administration may involve administration to multiple osteoarthritic joints. For example, i.a. administration to either or both knee or hip joints, or to a plurality of different types of joint, such as to one or more knee joints and to one or more hip joints. The administration to both knee joints may occur at substantially the same point in time, such as during the same appointment with the physician. The administration may involve ultrasound or fluoroscopy guided injection. In some cases, intra-articular administration does not comprise administration of a fusion protein comprising LINK_TSG6 or administration of a LINK_TSG6 nucleic acid for expression in a joint.

In some aspects described herein, the administration is not intrathecal.

Administration may be one time (once) per week. The week is preferably a calendar week (i.e. one administration on average every 7 days). Administration may be one time per month (one time per calendar month, or one administration on average every 30 days). In some preferred cases, the administration is monthly (1 administration on average every 30 days). In some cases, the treatment comprises administering LINK_TSG6 less frequently than once per week, such as once every two weeks, once every three weeks, or once per month. As used herein, the term “average” refers to the mathematical mean.

In some cases, one administration is given on average every 5, 6, 7, 8, 9, 10, 11, 12, 13, 14, 15, 16, 17, 18, 19, 20, 21, 22, 23, 24, 25, 26, 27, 28, 29, 30 or 31 days. Preferably, one administration is given on average every 7, 14, 21 or 30 days. In some cases, administration is given on average every 30 days.

Evaluation of a subject for osteoarthritic pain may occur any point before, during or after administration of a therapeutically effective amount of LINK_TSG6.

### Dosages

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

As disclosed herein, LINK_TSG6 was effective at reducing osteoarthritic pain when administered subcutaneously or intra-articularly, at both high and low dosages.

Several methods for determining the human equivalent dose (HED) based on murine dosages are known in the art and may be used to determine dosages suitable for use in the methods, treatments and compositions described herein.

In certain aspects, and particularly for subcutaneous administration, the dosage may be scaled for human administration based on body surface area comparisons for mouse and human. Alternatively, and particularly for intra-articular administration, the dosage may be scaled based comparative weights and drug concentrations or on “virtual space” considerations. In particular, see Nair and Jacob (Journal of basic and clinical pharmacy. 2016; 7(2), 27-31), WO2015085706A and Emami et al. 2018, the contents of which are incorporated herein in their entirety.

Thus, in some aspects, the treatment involves administration of 3 mg to 180 mg per month, 10 mg to 150 mg per month, 20 mg to 140 mg per month, 30 mg to 130 mg per month, 40 mg to 120 mg per month or 50 mg to 110 mg per month. In some aspects, the treatment involves subcutaneous administration of about 50 mg per month, about 60 mg per month, about 70 mg per month, about 80 mg per month, about 90 mg per month or about 100 mg per month of LINK_TSG6.

In some aspects described here, the treatment involves subcutaneous administration of 3 mg to 180 mg per month, 10 mg to 150 mg per month, 20 mg to 140 mg per month, 30 mg to 130 mg per month, 40 mg to 120 mg per month or 50 mg to 110 mg per month. In some aspects, the treatment involves subcutaneous administration of about 2 mg per month, about 3 mg per month, about 4 mg per month, about 5 mg per month, about 10 mg per month, about 20 mg per month, about 30 mg per month, about 40 mg per month, about 50 mg per month, about 60 mg per month, about 70 mg per month, about 80 mg per month, about 90 mg per month or about 100 mg per month of LINK_TSG6.

Higher doses are likely to be required given that many OA patients are overweight or obese. Moreover, less frequent dosing than once per month (such as 1 dose per 8 weeks) may likely require higher doses.

For intra-articular administration, the dosage may be scaled based on the comparative weights of mice and humans based on the methods described in WO2015085706A.

In some aspects, the treatment involves administration of 0.5 mg to 15 mg, 1 mg to 14 mg, 2 mg to 13 mg, 3 mg to 12 g, 4 mg to 11 mg, 5 mg to 10 mg, 6 mg to 9 mg or about 8 mg LINK_TSG6 per week.

In some aspects, the treatment may comprise administration of 0.5 mg to 50 mg, 0.5 to 45 mg, 1 mg to 40 mg, 2 mg to 35 mg, 5 mg to 30 mg, 10 mg to 20 mg LINK_TSG6. For example, at least 0.5 mg, 1 mg, 2 mg, 3 mg, 4 mg, 5 mg, 10 mg, 15 mg, 20 mg, 25 mg, 30 mg, 35 mg, 40 mg or 45 mg LINK_TSG6 per week.

### Formulations

Medicaments and pharmaceutical compositions according to aspects of the present invention may be formulated for administration by a number of routes, including but not limited to, subcutaneous, intra-articular, parenteral, intravenous, intra-arterial, intramuscular, oral and nasal. The medicaments and compositions may be formulated in fluid or solid form. Fluid formulations may be formulated for administration by injection to a selected region of the human or animal body. For example, a fluid formation may be formulated for administration by subcutaneous injection to the abdomen.

Preferably, medicaments and pharmaceutical compositions according to aspects of the present invention are formulated for subcutaneous or intra-articular administration. In some cases, the LINK_TSG6 is formulated with one or more other agents known to be useful for the treatment of OA. For example, intra-articular formulations of LINK_TSG6 may additionally comprise one or more of a corticosteroid, hyaluronic acid, or platelet rich plasma (see Ayhan E. et al. (2014) World J Orthop. 5(3):351-361.doi:10.5312/wjo.v5.i3.351).

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset, loss, or change, of taste (ageusia) and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. LINK_TSG6 used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1: Mouse pMx Model

To test whether Link_TSG6 reduces direct pain caused directly by OA, a partial medial meniscectomy (pMx) (Knights, Gentry & Bevan, 2012-292 Pain 153, 281) was carried out in one hind knee joint of 10 week-old C57BL/6 male mice to produce progressive degenerative joint disease and accompanying evoked pain behaviours. Under general anaesthesia, with the leg held in an extended position a medial para-patellar skin incision (approximately 3 mm) and arthrotomy was performed using a scalpel blade. The collateral ligaments were left intact and the patellar was not displaced. With the aid of a light microscope and angled 23 gauge needle, the medial meniscus was freed from its attachments to the margin of the tibial plateau and approximately half of the medial meniscus was removed (˜1 mm of tissue). The skin incision was closed with a wound closure clip. Sham operated animals underwent the same skin incision and arthrotomy procedures, but the meniscus was left intact. Link_TSG6 was administered (in PBS; ‘vehicle’) by sub-cutaneous (s.c.; at the nape of the neck) or intra-articular (i.a) injection 1×/week or 1×/month for 12 weeks commencing 1 week after surgery at a high (220 μg s.c./month; 40 μg i.a./month) or low (55 μg s.c./month; 10 μg i.a./month) dose. Vehicle was administered to control groups using the same routes and frequencies. All groups comprised 10 mice. Pain responses (on knee joint flexion, referred mechanical/thermal sensitivities in the paw and weight bearing on the operated leg) were assessed every week over the 12-week treatment period. Data were analysed using 1-way ANOVA with Tukey's post hoc test (* p<0.05, ** p<0.01, *** p<0.001).

### Example 2: Link_TSG6 Reduces Direct OA Joint Pain

Knee joint flexion was used to assess direct pain in the arthritic or sham-operated knee joint. Mice were gently restrained with one hand. With the thigh in a fixed position the thumb and forefinger of the investigator were used to flex and extend the knee joint through its full range of motion, passively 10 times. The number of audible vocalisations for each limb was recorded.

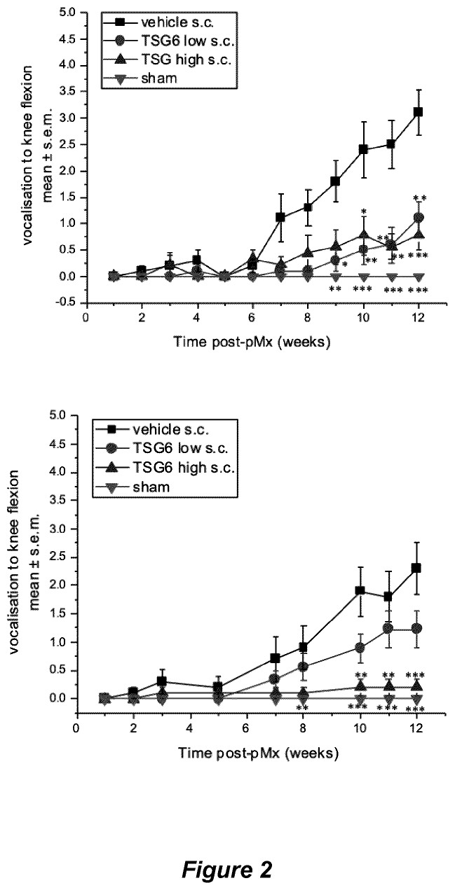

As shown by FIG. 2, sub-cutaneous administration of Link_TSG6 (high dose 1×/month) reduces direct OA joint pain in the mouse pMx model (assessed by vocalisations on flexion of the arthritic knee) to sham levels. Low and high doses 1×/week also have a significant treatment effect.

### Example 3: Link_TSG6 Reduces Thermal Sensitivity

Cold sensitivity was used to assess referred pain to the paw by the method described in (Gentry, Stoakley et al. (2010) Mol Pain 6:4) using a commercially available cold-plate (Ugo Basile; Milan, Italy). The cold-plate was set according to pre-determined calibration data using a surface temperature probe to correlate set temperature to actual surface temperature. The cold plate was allowed to stabilize at the set temperature for 5 minutes prior to testing. Paw withdrawal latencies were determined with the cold-plate set at 10° C. The animals were lightly restrained and each hind paw in turn placed onto the surface of the cold-plate. The end point was taken as the withdrawal of the paw and recorded as the withdrawal latency for the ipsilateral (operated leg) or the contralateral (un-operated leg) paw in seconds. A maximum cut-off of 30 seconds was used for each paw.

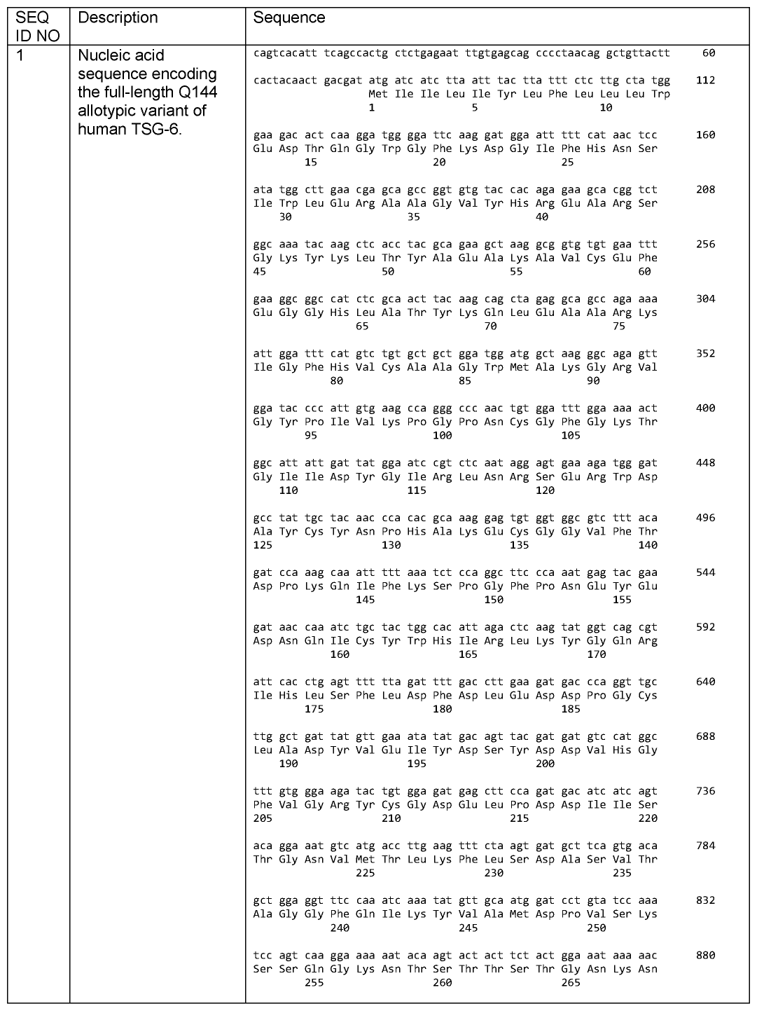

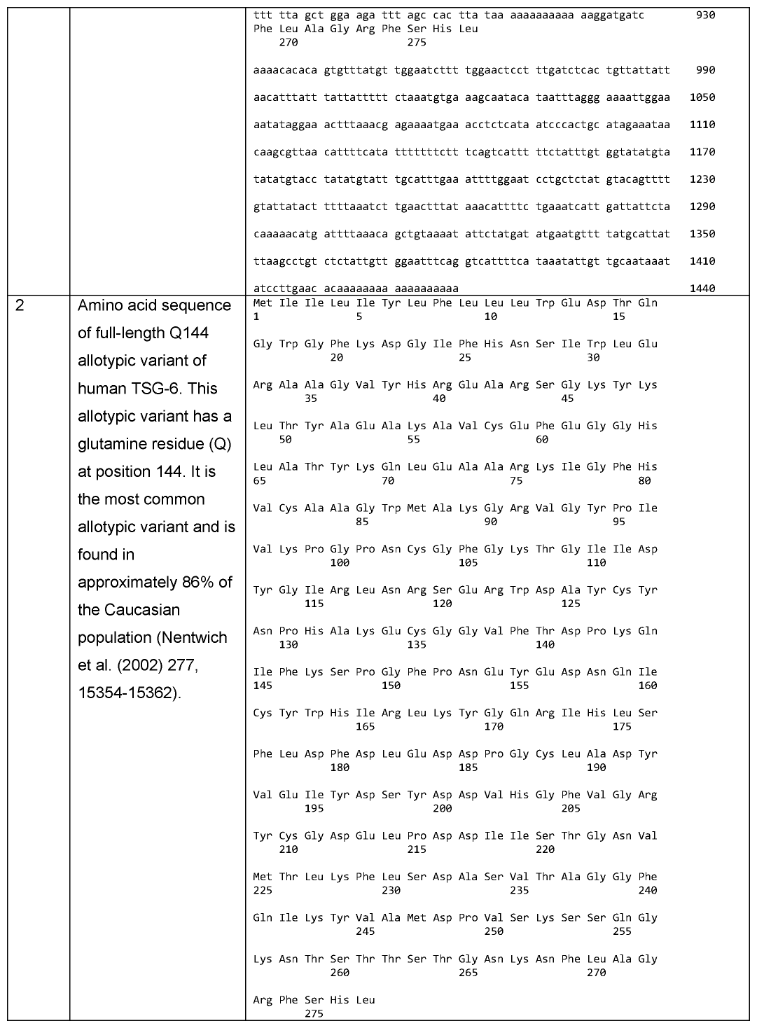

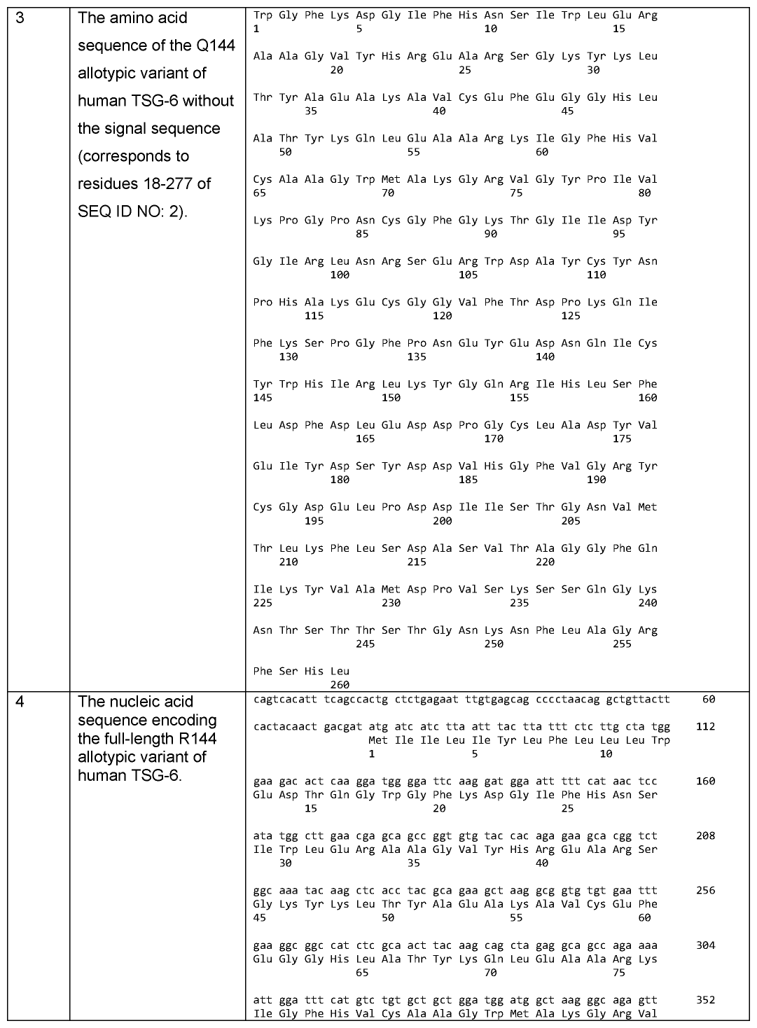

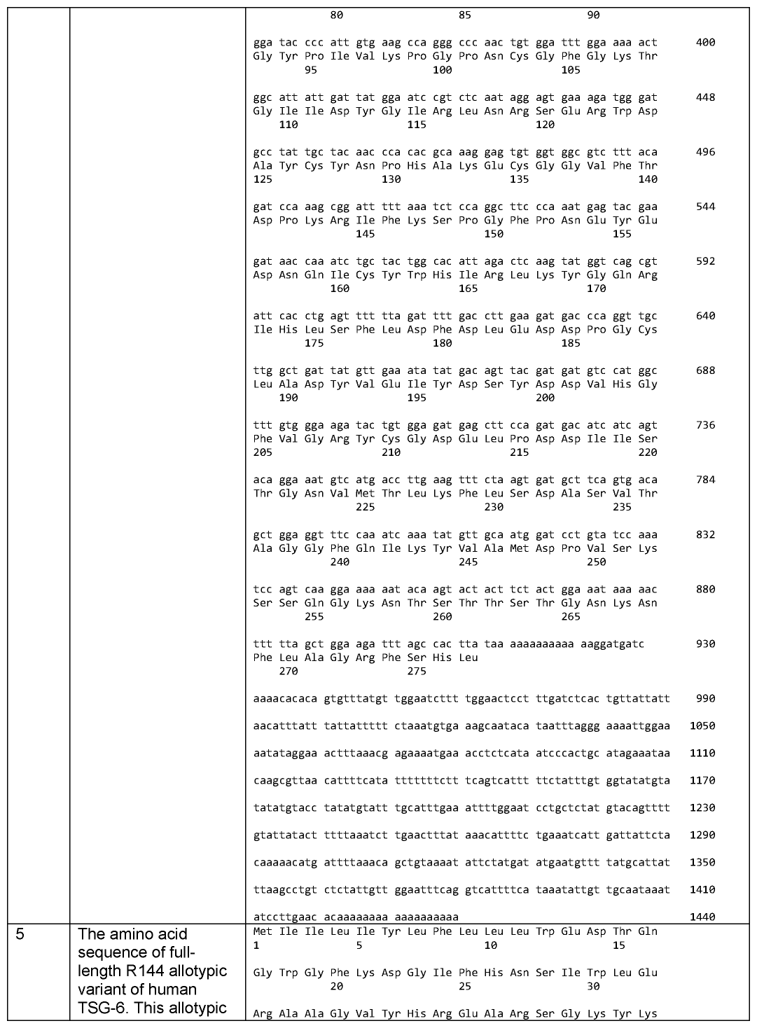

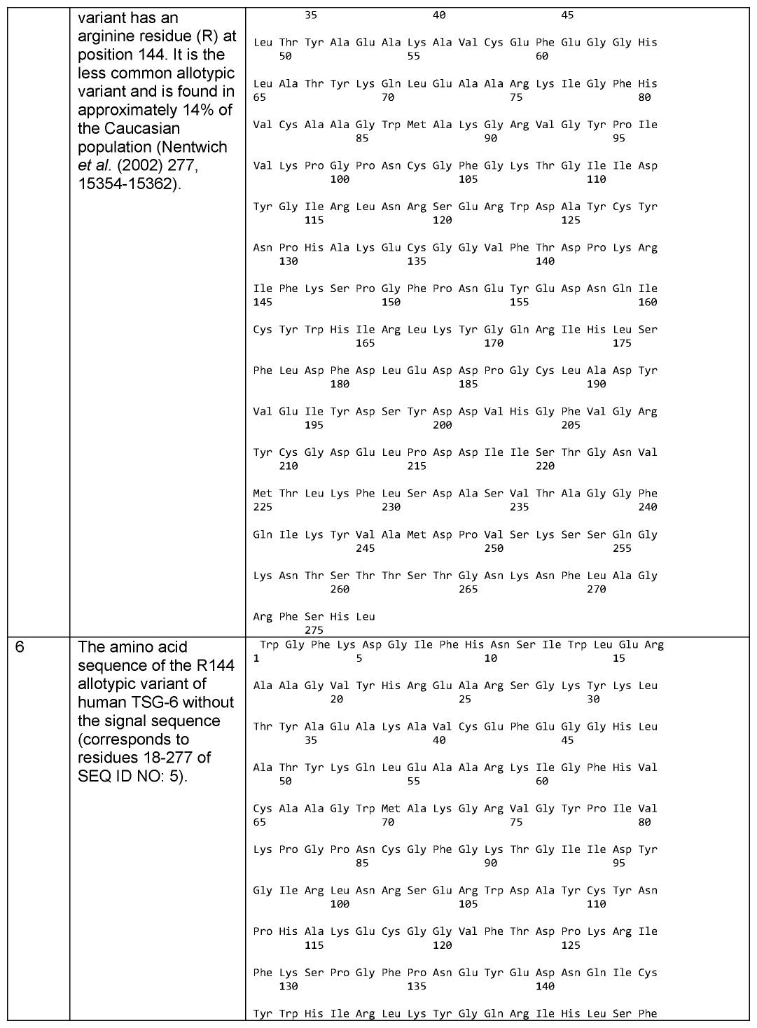

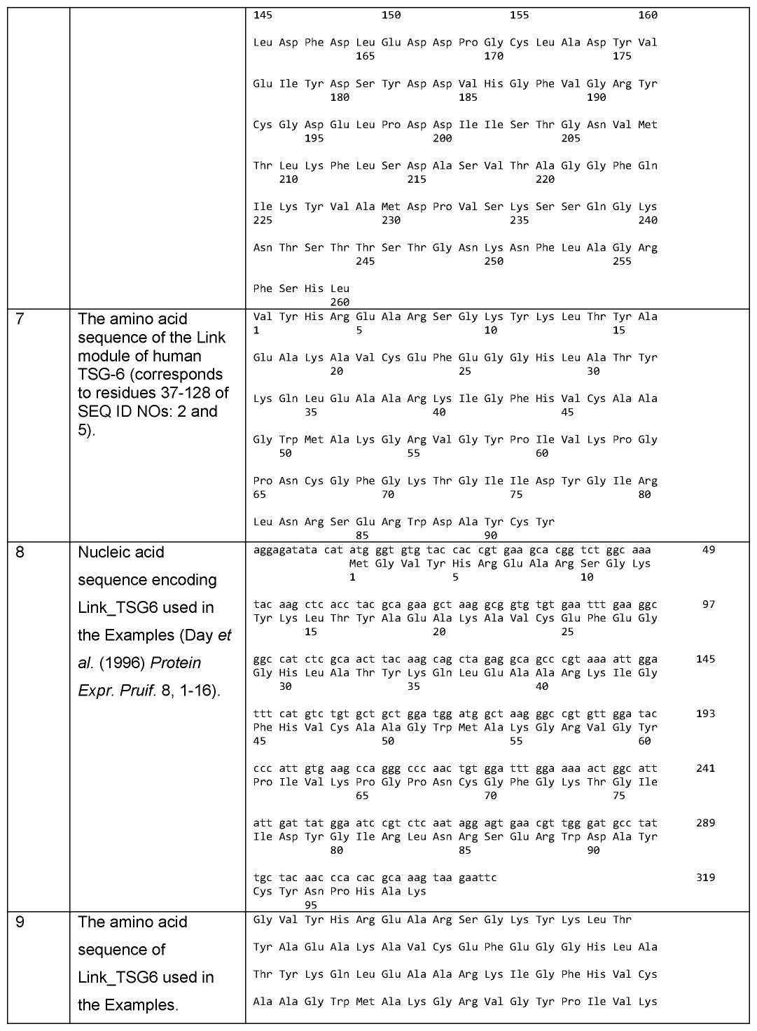

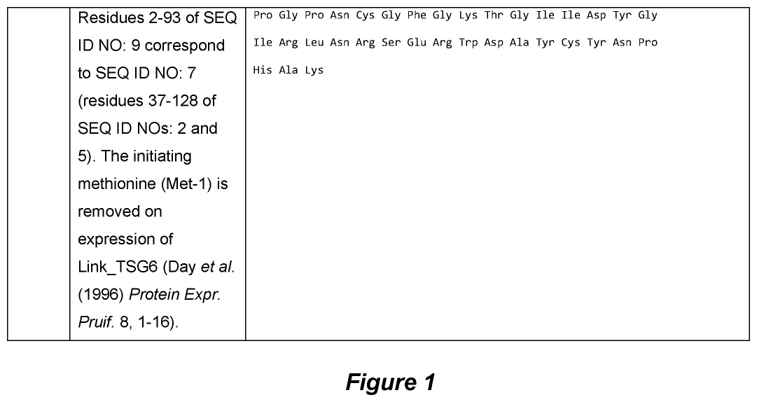

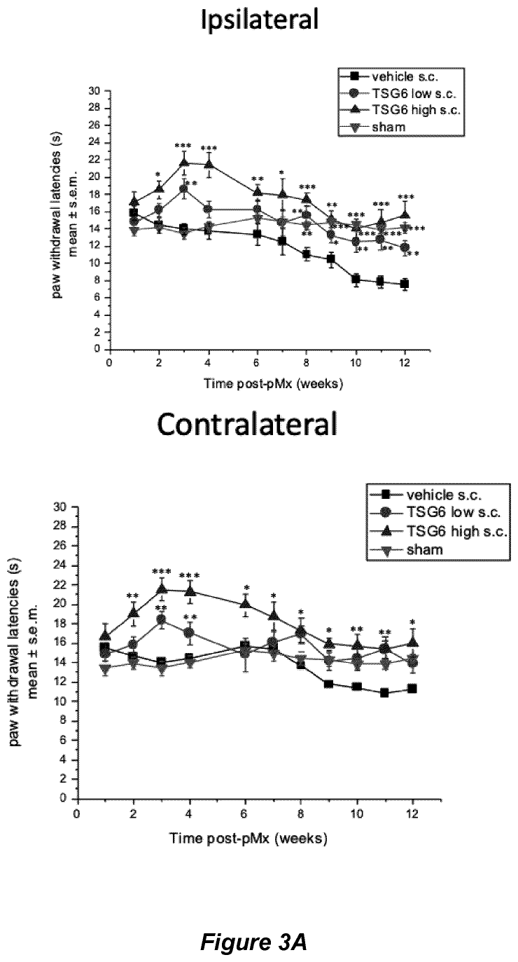

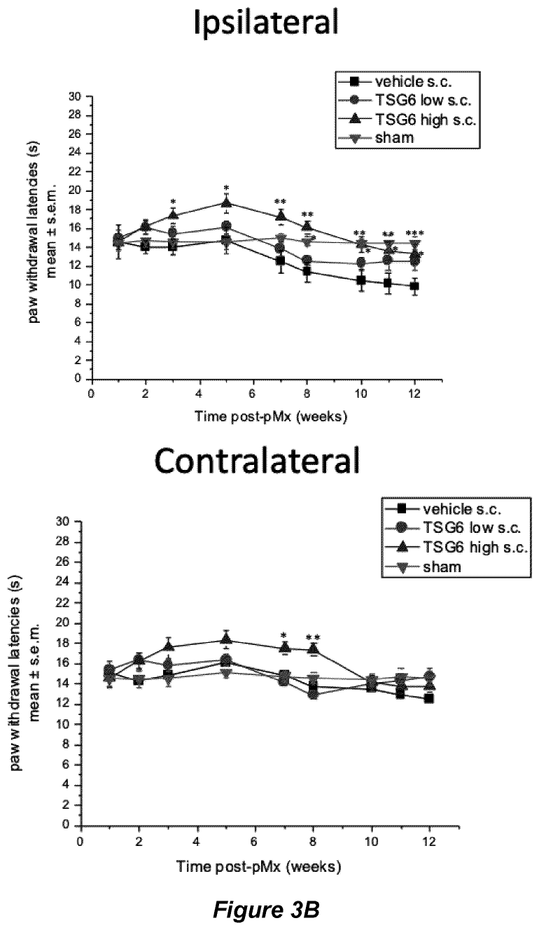

Sub-cutaneous Link_TSG6 treatment reduces thermal sensitivity in a mouse pMx model (assessed by paw withdrawal thresholds to a cold (10° C.) stimulus) in a dose-dependent manner, where the 1×/week (FIG. 3A) and 1×/month (FIG. 3B) treatments give similar effects in the ipsilateral (surgically-operated) paw. Here thermal sensitivity is reduced below sham levels indicating that Link_TSG6 is having an analgesic effect, acting at least in part, through the CNS. Consistent with this a significant analgesic effect is also apparent in the contralateral paw in both 1×/week (FIG. 3A) and 1×/month (FIG. 3B) treatments.

### Example 4: Link_TSG6 Reduces Mechanical Sensitivity

Mechanical hyperalgesia, as an indicator of referred pain to the paw, was assessed by measuring paw withdrawal threshold to an increasing pressure stimulus placed on the dorsal surface of the hind paw (Randall and Selitto (1957) Arch. Int. Pharmacodyn. CXI (4): 409-419) using an analgesymeter (model 7200, Ugo Basile; Milan, Italy) employing a dome shaped probe and a cut off of 150 grams. Paw withdrawal thresholds (in grams) were measured on ipsilateral and contralateral paws.

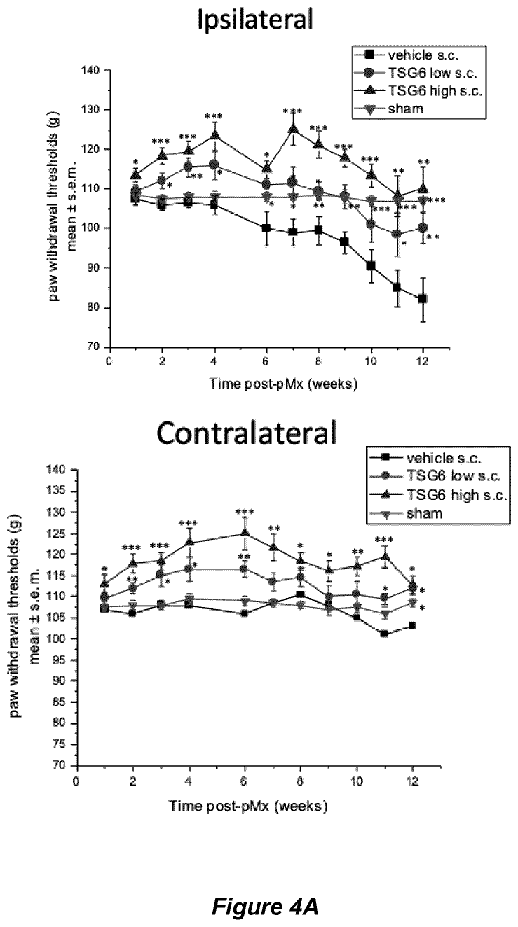

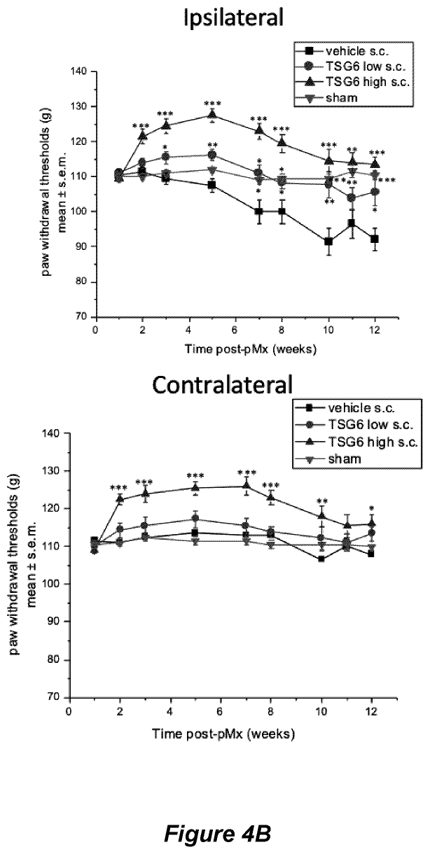

Sub-cutaneous Link_TSG6 treatment reduces mechanical sensitivity (assessed by paw withdrawal thresholds to mechanical pressure) in a dose-dependent manner, where the 1×/week (FIG. 4A) and 1×/month (FIG. 4B) treatments give similar effects in the ipsilateral (surgically-operated) paw. Here mechanical sensitivity is reduced below sham levels indicating that Link_TSG6 is having an analgesic effect, acting at least in part, through the CNS. Consistent with this a significant analgesic effect is also apparent in the contralateral paw in both 1×/week (FIG. 3A) and 1×/month (FIG. 3B) treatments.

### Example 5: Link_TSG6 Improves Weight Bearing in a Dose-Dependent Manner

The difference in weight borne by the ipsilateral compared to the contralateral hind limb was measured using a Dual Channel Weight Averager (Linton Instrumentation; Diss, Norfolk, UK). Mice were individually placed into a Perspex chamber designed to force them onto their hind limbs with each limb resting on a separate transducer pad. The transducer pad records the distribution of the animal's body weight on each paw over a 3 second period. Animals were allowed to acclimatise to the equipment on at least 2 occasions prior to taking the measurements. Results are presented as weight bearing difference (contralateral minus ipsilateral), in grams.

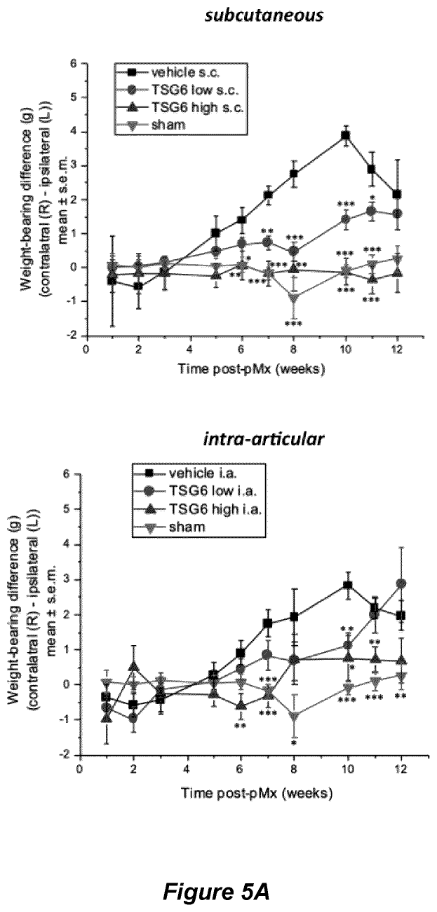

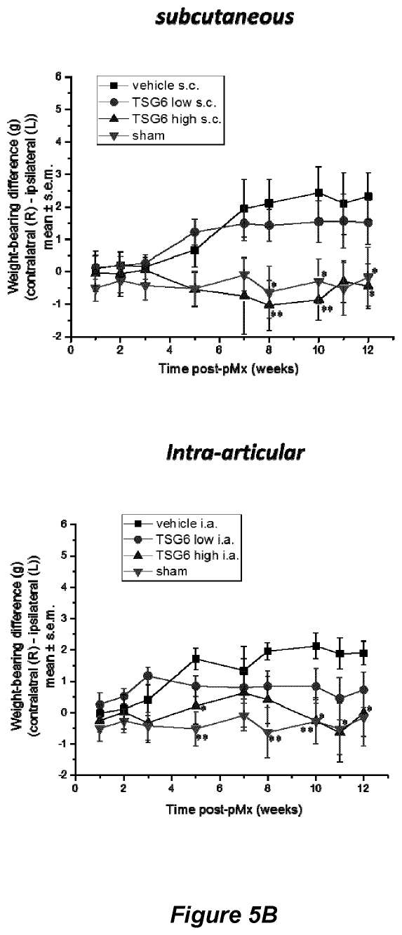

Link_TSG6 (1×/week) dose-dependently improves weight bearing (a combination of direct and referred pain effects) on the ‘arthritic’ (operated) leg (FIG. 5A). This effect was observed using subcutaneous (top) and intra-articular delivery (bottom). A dose-dependent effect of Link_TSG6 on weight bearing was also seen with 1×/month administration (FIG. 5B), where the high dose (s.c.) maintained weight bearing difference close to sham levels throughout the 12-week study period and intra-articular administration of the high dose also mediated significant effects reaching sham levels at the later time points.

### Example 6: Link_TSG6 Suppresses Tactile Allodynia in the ACLTpMMx Model

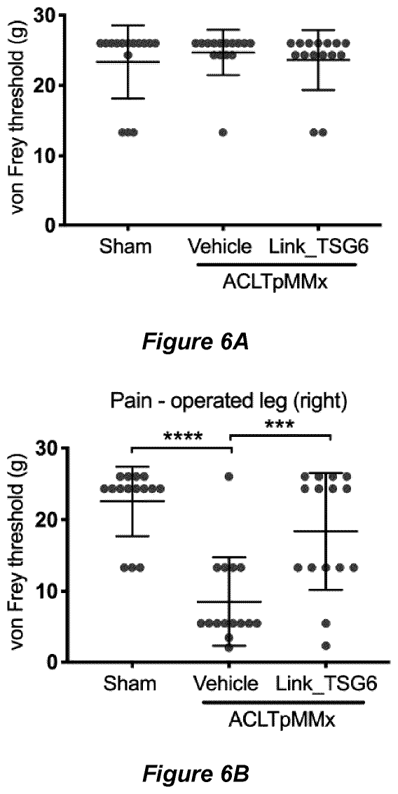

We investigated whether reduced joint damage in Link_TSG6-treated animals was associated with effects on a symptom of OA. ACLTpMMx rats (12-week old, male Lewis rats with OA induced by anterior cruciate ligament transection with partial (30%) medial meniscectomy) were treated with Link_TSG6 (40 μg/week, i.a.) and, at the 4-week time point, touch-evoked pain (tactile allodynia) was assessed in the paws of surgically destabilized (right) and un-operated control (left) legs as an indicator of secondary hyperalgesia. Paw withdrawal responses for the un-operated legs (in the absence or presence of Link_TSG6 treatment) were essentially identical to those of sham operated control animals (FIG. 6A). However, in ACLTpMMx-operated legs (vehicle treated) there was a significant increase in sensitivity to punctate mechanical stimulus. This response was significantly reduced by intra-articular injection of Link_TSG6 (FIG. 6B). There was no correlation between joint lesion area and the degree of tactile allodynia (data not shown). This is consistent with human OA where a significant proportion of individuals with extensive radiographic damage do not experience pain.

### Example 7: Methods to Extrapolate Intra-Articular Dosing from Mouse to Human

Several methods have been proposed in the art for extrapolating murine intra-articular doses to human. In particular, WO2015085706 describes a method based on comparative weights and drug concentrations, and Emami et al. (2018, Int J Toxicol. 37(1): 4-27) describe a method based on virtual space considerations.

### 7.1 Dosages Based on Comparative Weights

Equivalent human doses were calculated using the comparative weight based method, as described in WO2015085706. Human dosing can be extrapolated from animal dosing based on established relationships between metabolism, body size and weight in different species. It is therefore possible to scale up dosing from one species to another using the ratio of weights to the ¾ power, using the formula:

$$Dose ( human ) = Dose ( mouse ) \times \frac{{[ weight human ]}^{0.75}}{{[ weight mouse ]}^{0.75}}$$

As described in WO2015085706A, a typical human may be considered to be 70 kg. The mice used in our study may be considered to weigh around 20 g each.

Using the above formula and based on these weights, the monthly intra-articular low dose of 10 μg/month may correspond to approximately 4.56 mg per human per month:

$$4 , 555 μ g ( 4.56 mg ) = 10 μ g \times \frac{{[ 70 ]}^{0.75}}{{[ 0.02 ]}^{0.75}}$$

Based on these weights, the monthly intra-articular high dose of 40 μg/month may correspond to approximately 18.2 mg per human per month:

$$18 , 202 μ g ( 18.2 mg ) = 40 μ g \times \frac{{[ 70 ]}^{0.75}}{{[ 0.02 ]}^{0.75}}$$

Based on these calculations, a low dose may be 1.14 mg per human per week or a high dose may be 4.55 mg per human per week. Less frequent dosing, such as 1× per 8 weeks may require higher doses.

A typical injection volume for use in humans may be considered to be 2-10 ml (see WO2015085706A). Therefore, we calculated typical drug concentrations that may be efficacious in human subjects as follows:

For 2 ml injection: Low dose (monthly)=2.28 mg/ml; High dose (monthly)=9.11 mg/ml

For 10 ml injection: Low dose (monthly)=0.46 mg/ml; High dose (monthly)=1.82 mg/ml

For 2 ml injection: Low dose (weekly)=0.57 mg/ml; High dose (weekly)=2.28 mg/ml

For 10 ml injection: Low dose (weekly)=0.114 mg/ml; High dose (weekly)=0.46 mg/ml

We have performed calculations for 70 kg, but higher doses are likely to be required given that many OA patients are overweight or obese. We therefore calculated doses that were scaled on the basis of other weights; 60 kg, 80 kg, 90 kg, 100 kg, 110 kg and 120 kg to generate a range of values.

Example monthly doses were calculated with human weights of: 60 kg (low dose 4.05 mg; high dose 16.21 mg); 80 kg (low 5.03 mg; high 20.12 mg); 90 kg (low 5.50 mg; high 21.98 mg); 100 kg (low 5.95 mg; high 23.78 mg); 110 kg (low 6.39 mg; high 25.55 mg) and 120 kg (low 6.82 mg; high 27.27 mg).

Therefore, monthly dose ranges based on our mouse low and high dose data and human weight extrapolation may be 4.05 mg-27.27 mg. Weekly dose ranges based on our mouse low and high dose data and human weight extrapolation may be 1.01 mg-13.64 mg. Less frequent than 1×/month dosing would allow for higher doses to be extrapolated.

### 7.2 Dosages Based on “Virtual Space” Considerations.

Human equivalent doses for intra-articular administration were calculated using the virtual space method (based on Emami et al. Int J Toxicol. 2018; 37(1): 4-27. doi:10.1177/1091581817741840). The virtual space method takes into account differences in the size of the intra-articular space between mouse and human, and scales the dose so that the drug concentration within the respective joint spaces is similar. A hypothetical example depicting this method of dose scaling is given in Emami et al., 2018; FIG. 7.

Calculating equivalent doses based on virtual space considerations is based on the assumption that the administration site (such as intra-articular spaces in joints) are virtual fluid filled chambers into which the drug product is introduced and diffuses throughout, reaching a steady state concentration at which the drug resides for some time before eventually being cleared from the area. Therefore, the size of the virtual space in the mouse OA model was estimated, and then scaled to the virtual space into which the drug will be administered, i.e. the virtual space in the joint of an OA patient. As we don't know the steady state concentration of Link_TSG6 in synovial fluid (in mouse or human) we have used the concentration at time of injection to scale from mouse to human doses and assumed kinetics of clearance and steady state values are the same in these two species.

To estimate virtual space in the mouse model, mouse synovial fluid volume was considered to be 3-5 μl (based on Xu et al., (2017). Bone Res. 5: 17012). Human synovial fluid volume in OA patients was considered to be between 0.56 and 71.71 ml (median 3.05 ml), (taken from Kraus et al. (2007). Osteoarthritis & Cartilage. 15; 1217-1220). Therefore for illustratory calculations we assumed mouse SF volume may be 4 μl and human SF volume in OA patients may be 3 ml. For our pMx study we injected 5 μl in to the mouse knee joint per injection. We calculated equivilant dosages based on a 2-10 ml injection volume which may be considered typically used in humans (see WO2015085706A patent text).

### 7.2.1 Monthly Doses

If we assume a synovial fluid volume (SF) of 4 μl SF in our mice, to which we adminstered a 5 μl injection, the total volume is 9 μl. Our low intraarticular dose was 10 μg/month, administered to total volume of 9 μl, or 1.11 mg/ml. The high intraarticular was 40 μg/month, administered in 9 μl total volume, or 4.44 mg/ml.

As mentioned above, the SF volume is known to vary in OA patients, between 0.56 and 71.71 ml (median 3.05 ml). Taking the median value of approximately 3 ml SF, a 2 ml injection would give a total volume of 5 ml, meaning that for a dosage of 1.11 mg/ml concentration, a 2 ml injection of 2.78 mg/ml (5.56 mg) would be required or a 1 ml injection of 1.44 mg/ml (14.4 mg), for the equivalent low dose in human.

For the higher doses equivalent to 4.44 mg/ml, a 2 ml injection of 11.1 mg/ml or a 10 ml injection of 5.77 mg/ml would be required (i.e. 22.2 mg in 2 ml or 57.7 mg in 10 ml).

For different volumes of human SF it is possible to scale accordingly. We also calculated dose ranges for different human SF volumes, for 2 and 10 ml injection volumes.

| | | | | Solution | |
|---|---|---|---|---|---|
| Human | | | Concen- | concen- | |
| SF | Injection | Total | tration | tration for | |
| volume | volumne | volume | in SF | injection | Adminstered |
| (ml) | (ml) | (ml) | (mg/ml) | (mg/ml) | dose (mg) |
| 10 | 2 | 12 | 1.11 (low) | 6.66 | 13.32 |
| 10 | 10 | 20 | 1.11 (low) | 2.22 | 22.2 |
| 10 | 2 | 12 | 4.44 (high) | 26.64 | 53.28 |
| 10 | 10 | 20 | 4.44 (high) | 8.88 | 88.8 |
| 1 | 2 | 3 | 1.11 (low) | 1.67 | 3.34 |
| 1 | 10 | 11 | 1.11 (low) | 1.22 | 12.2 |
| 1 | 2 | 3 | 4.44 (high) | 6.68 | 13.36 |
| 1 | 10 | 11 | 4.44 (high) | 4.88 | 48.8 |
| 30 | 2 | 32 | 1.11 (low) | 17.76 | 35.52 |
| 30 | 10 | 40 | 1.11 (low) | 4.44 | 44.4 |
| 30 | 2 | 32 | 4.44 (high) | 71.04 | 142.08 |
| 30 | 10 | 40 | 4.44 (high) | 17.76 | 177.6 |

### 7.2.2 Weekly Doses

If we assume a synovial fluid volume (SF) of 4 μl SF in our mice, to which we adminstered a 5 μl injection, the total volume is 9 μl. Our low intraarticular dose was 2.5 μg/week, administered to total volume of 9 μl, or 0.28 mg/ml. The high intraarticular was 10 μg/month, administered in 9 μl total volume, or 1.11 mg/ml.

Applying the methodology outlined above at 6.2.1, the equivalent weekly dose for humans is calculated as follows:

| | | | | Solution | |
|---|---|---|---|---|---|
| Human | | | Concen- | concen- | |
| SF | Injection | Total | tration | tration for | |
| volume | volumne | volume | in SF | injection | Adminstered |
| (ml) | (ml) | (ml) | (mg/ml) | (mg/ml) | dose (mg) |
| 3 | 2 | 5 | 0.28 (low) | 0.70 | 1.40 |
| 3 | 10 | 13 | 0.28 (low) | 0.36 | 3.60 |
| 3 | 2 | 5 | 1.11 (high) | 2.78 | 5.56 |
| 3 | 10 | 13 | 1.11 (high) | 1.44 | 14.4 |
| 10 | 2 | 12 | 0.28 (low) | 1.68 | 3.37 |
| 10 | 10 | 20 | 0.28 (low) | 0.56 | 5.6 |
| 10 | 2 | 12 | 1.11 (high) | 6.66 | 13.32 |
| 10 | 10 | 20 | 1.11 (high) | 2.22 | 22.2 |
| 1 | 2 | 3 | 0.28 (low) | 0.48 | 0.84 |
| 1 | 10 | 11 | 0.28 (low) | 0.31 | 3.05 |
| 1 | 2 | 3 | 1.11 (high) | 1.67 | 3.34 |
| 1 | 10 | 11 | 1.11 (high) | 1.22 | 12.2 |
| 30 | 2 | 32 | 0.28 (low) | 4.44 | 8.88 |
| 30 | 10 | 40 | 0.28 (low) | 1.11 | 11.1 |
| 30 | 2 | 32 | 1.11 (high) | 17.76 | 35.52 |
| 30 | 10 | 40 | 1.11 (high) | 4.44 | 44.4 |

### 7.2.3 Dose Ranges

Equivalent dose ranges for SF volumes of 1-30 ml (and injection volumes of 2 or 10 mls) based on our mouse low and high dose data may therefore be 3.34 mg-177.6 mg per month. Weekly dose ranges for SF volumes of 1-30 ml (and injection volumes of 2 or 10 ml) based on our mouse low and high dose data may therefore be 0.84 mg-44.4 mg. Less frequent than 1×/month dosing would allow for higher doses to be extrapolated.

### REFERENCES

A number of publications are cited above in order to more fully describe and disclose the invention and the state of the art to which the invention pertains. Full citations for these references are provided below. The entirety of each of these references is incorporated herein.
Day A. J. & Milner C. M. TSG-6: A multifunctional protein with anti-inflammatory and tissue-protective properties. Matrix Biology. 2019; 78-79:60-83. doi:10.1016/j.matbio.2018.01.011.
Altman R. D. & Barthel H. R. Topical therapies for osteoarthritis. Drugs. 2011; 71(10):1259-1279. doi:10.2165/11592550-000000000-00000
Kompel A. J., Roemer F. W., Murakami A. M., Diaz L. E., Crema M. D., Guermazi A. Intra-articular Corticosteroid Injections in the Hip and Knee: Perhaps Not as Safe as We Thought?. Radiology. 2019; 293(3):656-663. doi:10.1148/radiol.2019190341
Yang, H., Wu, L., Deng, H., Chen, Y., Zhou, H., Liu, M., Wang, S., Zheng, L., Zhu, L., & Lv, X. Anti-inflammatory protein TSG-6 secreted by bone marrow mesenchymal stem cells attenuates neuropathic pain by inhibiting the TLR2/MyD88/NF-κB signaling pathway in spinal microglia. Journal of neuroinflammation, 2020; 17(1), 154.
Lo G., McAlindon T., Niu J., Zhang Y., Beals C., Dabrowski C, et al. Strong Association of Bone Marrow Lesions and Effusion with Pain in Osteoarthritis. Arthritis & Rheumatism. 2008; 56(9):S790. Ref Type: Abstract.
McAlindon T., Formica M., Schmid C. H., Fletcher J. Changes in barometric pressure and ambient temperature influence osteoarthritis pain. Am J Med. 2007; 120(5):429-434.
Riddle, D. L., Wade, J. B., Jiranek, W. A., & Kong, X. Preoperative pain catastrophizing predicts pain outcome after knee arthroplasty. Clinical orthopaedics and related research. 2010; 468(3), 798-806.
Knights, C., Gentry, C. & Bevan, S. Partial medial meniscectomy produces osteoarthritis pain-related behaviour in female C57BL/6 mice. Pain. 2012; 153. 281-92. 10.1016/j.pain.2011.09.007.
Gentry C., Stoakley N., Andersson D. A., Bevan S. The Roles of iPLA2, TRPM8 and TRPA1 in Chemically Induced Cold Hypersensitivity. Molecular Pain. 2010. doi:10.1186/1744-8069-6-4.
Randall L. O, Selitto J. J. A method for measurement of analgesic activity on inflamed tissue. Arch Int Pharmacodyn Ther. 1957; 111(4):409-419.
Ichiseki T., Shimazaki M., Ueda Y, et al. Intraarticularly-Injected Mesenchymal Stem Cells Stimulate Anti-Inflammatory Molecules and Inhibit Pain Related Protein and Chondrolytic Enzymes in a Monoiodoacetate-Induced Rat Arthritis Model. Int J Mol Sci. 2018; 19(1):203. Published 2018 Jan. 9.
Day A. J., Drummond S. P., Price A., Anand S., Bartnik. E. and Milner C. M. A novel chondroprotective property of TSG-6 has therapeutic potential for OA. Osteoarthritis and Cartilage. 2016; 24:S19-S20. Ref Type: Abstract.
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A method of treating osteoarthritic pain comprising administering LINK_TSG6 to a patient in need thereof.

2. The method of claim 1, wherein the LINK_TSG6 is administered subcutaneously or intra-articularly.

3. The method of claim 1, wherein the LINK_TSG6 is administered once per week or less frequently.

4. The method of claim 1 wherein the osteoarthritic pain is referred pain.

5. The method of claim 3, wherein the LINK_TSG6 is administered once per 7 days, once per 10 days, once per 14 days, once per 21 days, once per 30 days, once per 45 days, once per 60 days, once per 90 days, or less than once per 90 days.

6. The method of claim 1, wherein the LINK_TSG6 polypeptide comprises, consists, or consists essentially of (i) the amino acid sequence of SEQ ID NO: 7 or SEQ ID NO: 9, or (ii) an amino acid sequence having at least 80% identity to the amino acid sequence of SEQ ID NO: 7 or SEQ ID NO: 9.

7. A composition comprising LINK_TSG6, wherein the composition is formulated for subcutaneous administration.

8. The composition according to claim 7, wherein the composition comprises 10 mg to 150 mg of LINK_TSG6 per dose.

9. A composition comprising LINK_TSG6, wherein the composition is formulated for intra-articular administration.

10. The composition according to claim 9, wherein the composition comprises 1 mg to 180 mg of LINK_TSG6 per dose.

11. (canceled)

12. (canceled)

13. The method of claim 1 wherein the method comprises subcutaneous administration of 10 mg to 150 mg of LINK_TSG6 to the patient.

14. The method of claim 1 wherein the method comprises intra-articular administration of 1 mg to 180 mg of LINK_TSG6 to the patient.
